(19) United States
(12) Patent Application Publication
(10) Pub. No.: US 2026/0314997 A1
(43) Pub. Date: Oct. 15, 2026

(54) SELF-ADMINISTERED CARPAL TUNNEL SYNDROME ASSESSMENT

(71) Applicant: Optimum Upper Extremity SAS, Lewisburg, PA (US)

(72) Inventors: Louis Christopher Grandizio, Lewisburg, PA (US); Joel Christian Klena, Lewisburg, PA (US)

(21) Appl. No.: 19/647,443

(22) Filed: Apr. 14, 2026

Related U.S. Application Data
(60) Provisional application No. 63/789,181, filed on Apr. 15, 2025.

(51) Int. Cl. A61B 5/00; G16H 10/20

(52) U.S. Cl. CPC A61B 5/459; A61B 5/0002; G16H 10/20

(57) ABSTRACT

The present disclosure provides a computer-implemented method of guideline-directed self-assessment of CTS using a set of CTS symptoms. The method may include for each CTS symptom in the set of CTS symptoms: displaying a CTS symptom self-assessment guideline and prompting a patient to perform the guideline and observe results; displaying information regarding how the results correlate with whether the patient does or does not exhibit the CTS symptom and prompting input of a yes/no indicator correlating with whether the patient exhibits the CTS symptom; receiving and storing an input indicator for the CTS symptom in a set of CTS symptom indicators, wherein the input indicator has a value of 1 for yes and a value of 0 for no; determining a CTS assessment for the patient, based on a comparison of a sum of the values of the CTS indicators to a cutoff point; and displaying the CTS assessment.

### TECHNICAL FIELD

The present disclosure provides a method for assessing carpal tunnel syndrome (CTS) that is self-administered by a patient. The method may involve the guideline-directed self-assessment of CTS symptoms by a patient at home or in another non-clinic/office setting. The method may allow patients to receive the results of the self-assessment directly. The method may further include providing a recommendation to the patient regarding referral to a medical professional or the need for repeated assessment or diagnostic procedures, based on self-assessed CTS symptoms. The method may also include transmitting the results to a medial professional. The present disclosure further provides a system for implementing such a method.

### BACKGROUND

Despite being the most common form of peripheral compressive neuropathy in the upper-extremity, diagnostic modalities for carpal tunnel syndrome (CTS) remain somewhat controversial. While CTS can be diagnosed with simple history and physical examination, electrodiagnostic studies (EDS) are frequently utilized in evaluating patients with suspected peripheral nerve compression (Colombo, Billig/Sears). In 2016, The American Academy of Orthopaedic Surgeons (AAOS) modified the clinical practice guidelines for CTS and no longer recommended routine EDS for all patients undergoing planned carpal tunnel release (CTR), and instead incorporated six history and physical examination items with a weighted scoring system (CTS-6) designed to be used by non-expert medical professionals. CTS-6 has demonstrated high levels of inter-rater reliability for non-expert physicians and when modified for use in telemedicine (Sears, Billig, Grandizio). Nevertheless, recent authors have noted that 67% of patients referred for subspeciality hand care still underwent EDS prior to their visit and many upper-extremity surgeons routinely require pre-consultation EDS (Colombo, Billig/Sears).

Physicians remain concerned about physical examination, screening and assessment performed outside of the traditional in-office setting (Geyer, Van Nest). This is particularly evident for telemedicine, where prior investigations have demonstrated that willingness to use these services are associated with both patient and physician preferences and comfort (Tinwala, Baker). The limited clinical investigations within upper-extremity surgery which have analyzed remote assessment have demonstrated mixed results with respect to diagnostic performance and have varied with respect to the type of technology utilized (video, images, etc) (Tripod, Orman, Scott, Grandizio). Despite numerous review articles detailing modified physical examination maneuvers for telemedicine, diagnostic performance in this context has also been infrequently assessed (Grandizio, Van Nest). In patients with peripheral compressive neuropathy, it remains uncertain how history and physical examination performs without direct physician supervision when compared to conventional, physician-administered history and examination.

Others have assessed unconventional diagnostic and screening tools for hand conditions that patients can administer without physician guidance. Hageman analyzed 86 patients who had used WebMD symptom checker to determine their assessment, nothing that only 33% of diagnoses matched the one determined by a hand surgeon. Koyama developed a smartphone-based CTS screening app administered to patients using an anomaly detection algorithm. This app demonstrated high sensitivity (94%), but specificity was only 64%. Artificial intelligence (AI) and machine-learning algorithms have been frequently discussed as a potential diagnostic alternative in the context of CTS. However, many of the existing AI-focused investigations have had study authors or physicians enter the recorded history and examination information or have required in the input of tests that patients cannot self-administer (Kuroiwa, Ardakani, Yener). Diagnostic performance results of these studies have been inconsistent.

Similarly, a version of the CTS-6 modified for use in telemedicine has been previously evaluated, and the individual components demonstrated agreement between 63% (2PD) and 100% (both history items) (Grandizio). However, in this prior study, the CTS-6 components administered via telemedicine were still physician guided.

### SUMMARY

The disclosure provides computer-implemented method of guideline-directed self-assessment of carpal tunnel syndrome (CTS) using a set of CTS symptoms, the method comprising: for each CTS symptom in the set of CTS symptoms: displaying a CTS symptom self-assessment guideline and prompting a patient to perform the guideline and observe results; displaying information regarding how the results correlate with whether the patient does or does not exhibit the CTS symptom and prompting input of a yes/no indicator correlating with whether the patient exhibits the CTS symptom; receiving and storing an input indicator for the CTS symptom in a set of CTS symptom indicators, wherein the input indicator has a value of 1 for a yes indicator and a value of 0 for a no indicator; determining a CTS assessment for the patient, based on a comparison of a sum of the values of the CTS indicators to a cutoff point; and displaying the CTS assessment to the patient or transmitting the CTS value to a physician associated with the patient.

The present disclosure further provides the above method with any of the following additional steps or features, which may be combined with one another in any compatible combinations:
the set of CTS symptoms comprises between 3-6 symptoms;
the set of CTS symptoms comprises 5 symptoms;
the CTS symptoms comprise or consist of: numbness in median nerve distribution; subjective night symptoms; thenar atrophy; phalen test; loss of 2-point discrimination; or tinel sign, and any combinations thereof;
the CTS symptoms comprise or consist of: numbness in median nerve distribution; subjective night symptoms; thenar atrophy; phalen test; or loss of 2-point discrimination, and any combinations thereof;
the CTS symptoms comprise or consist of: numbness in median nerve distribution; subjective night symptoms; or phalen test, and any combinations thereof;
the guideline comprises instructions for performing a physical evaluation; answering a question regarding current or recent physical state without the need for a physical evaluation; or observing results, and any combinations thereof;
the CTS assessment is yes/no assessment correlating with whether the patient does or does not have CTS;
the CTS assessment is a graduated assessment correlating with likelihoods that the patient has CTS;
the sum of the values of the CTS indicators is a simple sum of the values of the set of CTS symptom indicators;
the sum of the values of the CTS indicators is a weighted sum of the values of the set of CTS symptom indicators;
the CTS assessment is determined using a method that has an accuracy based on ROC of at least 75% when compared to in-clinic CTS diagnoses by physicians with CTS expertise;
the CTS assessment is determined using a method that has a sensitivity/specificity of at least 0.85 when compared to in-clinic CTS diagnoses by physicians with CTS expertise;
the CTS assessment is determined using a method that has a J value of at least 0.6 when compared to in-clinic CTS diagnoses by physicians with CTS expertise; the method further comprises, prior to displaying a CTS symptom self-assessment guideline, displaying a request for a patient ID associated with the patient, and receiving an input of the patient ID; the method further comprises, based on the CTS assessment, displaying a further medical care recommendation;
the CTS assessment correlates with the patient not having CTS, the further medical care recommendation comprises a recommendation that further medical care for CTS is not needed;
the CTS assessment correlates with the patient having CTS, or having a likelihood of having CTS above a selected level, the further medical care recommendation comprises a recommendation that the patient seek professional medical care for CTS;
the method further comprises displaying a referral recommendation comprising information regarding a medical professional with expertise in CTS; and
the method further comprises: requesting permission to transmit the CTS assessment and patient-identifying information to at least one medical professional from the referral recommendation; receiving an input granting or denying such permission; and if such permission is granted, transmitting the CTS assessment and patient-identifying information to the medical professional(s).

The present disclosure further provides a method of diagnosis of CTS in a patient by a physician, the method comprising: obtaining a CTS assessment of the patient, wherein the CTS assessment is generated by any of the above methods; and based on the CTS assessment, undertaking a diagnostic or therapeutic action.

The present disclosure further provides the above method with any of the following additional steps or features, which may be combined with one another in any compatible combinations:
the diagnostic action comprises a physical examination of the patient by a medical professional; assessment of the CTS symptoms using any of the above methods by or under direction of the medical professional; or diagnostic testing, and any combinations thereof; and
the therapeutic action comprises surgery to alleviate CTS; referral to another physician with expertise in CTS, or both.

The present disclosure further provides a method of seeking treatment or diagnosis of CTS by a patient, the method comprising:
obtaining a CTS assessment of the patient, wherein the CTS assessment is generated by the any of the above methods; and, based on the CTS assessment, determining whether to seek further medical care.

The present disclosure further provides a system for guideline-directed self-assessment of carpal tunnel syndrome (CTS) using a set of CTS symptoms, the system comprising: an electronic device comprising a processor, memory, display, input, and I/O interface capable of storing instructions within the memory that, when executed by the processor, cause the electronic device to perform or communicate via the I/O interface over a network with a patient data processing center that includes a processor, a memory and an I/O interface, and cause the patient data processing center to perform a self-administered CTS assessment method according to any of the above methods; or a patient data processing center comprising a processor, a memory, and an I/O interface capable of storing instructions within the memory that, when executed by the processor cause the patient data processing center to perform or communicate via the I/O interface over a network with an electronic device containing a processor, memory, display, and input and cause the electronic device to perform a self-administered CTS assessment method according to any of the above methods.

### BRIEF DESCRIPTION OF THE FIGURES

Aspects of the present disclosure may be better understood through reference to the following detailed description and figures, which represent embodiments of the disclosure.

FIG. 1 is a flowchart of a method for guideline-directed self-assessment of CTS.

FIG. 2 is a flowchart of a method for diagnosis of CTS in a patient by a physician.

FIG. 3 is a flowchart of a method for a patient to seek CTS diagnosis or treatment.

FIG. 4 is a ROC curve calculated using as a predictor the CTS total score calculated using a web-based CTS self-assessment method and the in-clinic CTS diagnosis for the same patient as the binary outcome. Area under the curve=0.916.

FIG. 5 is a ROC curve for predicted probabilities in a web-based CTS self-assessment method based on 6 CTS symptoms (S1-S6). Results were obtained using 70% of the patient sample.

FIG. 6 is a ROC curve for the simple sum score of 6 CTS symptoms (S1-S6) in a web-based CTS self-assessment method. Results were obtained using 70% of the patient sample.

FIG. 7 is a ROC curve for predicted probabilities in a web-based CTS self-assessment method based on 5 CTS symptoms (S1-S5). Results were obtained using 70% of the patient sample.

FIG. 8 is a ROC curve for the simple sum score of 5 CTS symptoms (S1-S5) in a web-based CTS self-assessment method. Results were obtained using 70% of the patient sample.

FIG. 9 is a ROC curve for predicted probabilities in a web-based CTS self-assessment method based on 3 CTS symptoms (S1, S2 and S4). Results were obtained using 70% of the patient sample.

FIG. 10 is a ROC curve for the simple sum score of 3 CTS symptoms (S1, S2 and S4) in a web-based CTS self-assessment method. Results were obtained using 70% of the patient sample.

FIG. 11 is a ROC curve for the weighted sum score of 6 CTS symptoms (S1-S6) in a web-based CTS self-assessment method.

FIG. 12 is a ROC curve for the weighted sum score of 6 CTS symptoms (S1-S5) in a web-based CTS self-assessment method.

FIG. 13 is a ROC curve for the weighted sum score of 3 CTS symptoms (S1, S2 and S4) in a web-based CTS self-assessment method.

### DETAILED DESCRIPTION

The present disclosure provides a method for assessing carpal tunnel syndrome (CTS) that is self-administered by a patient as directed by guidelines. The method may involve the guideline-directed self-assessment of CTS symptoms. The results of the assessment may be provided directly to the patient, allowing the patient to independently determine what sort of further care the would like to seek, or whether it is reasonable to not seek further care at that time. If the patient does seek further care, the results may help the patient make choices that better match their medical needs, such as seeking an appointment with a medical professional who is an expert in CTS only if CTS seems likely, and seeking appointments or testing with medical professionals who specialize in different hand/wrist conditions if CTS is not likely. Even if the patient does not seek further care, or continues with a course of care that is not for CTS, the patient may feel reassured that they are not likely to have an overlooked CTS condition.

The method may further include providing a recommendation to the patient regarding referral to a medical professional or the need for repeated assessment or diagnostic procedures, based on self-assessed CTS symptoms. The method may also transmit the results of the assessment to a physician or other medical professional, such as a medical professional who recommended that the patient perform the self-assessment, a medical professional otherwise already providing care to the patient, or a new medical professional, such as a medical professional to whom the patient is referred as part of the method.

The method may also include storing the results for the patient or a medical professional to view later, or for transmission to a medical professional selected later by the patient. Additionally, the method may include providing information about CTS, other hand/wrist conditions, the results, or a referred medical professional, for example by links to web pages, particularly reliable external web pages.

The present disclosure further provides a system for implementing such a method.

Methods and systems described herein are designed to allow a patient to determine whether they should seek a professional medical evaluation and treatment for CTS. By allowing patients to perform a CTS assessment in a simple and not time-consuming manner, the methods and systems may assist the patient in obtaining appropriate medical care more quickly or at less expense. The methods and systems may also encourage patients likely to have CTS to seek medical care when they otherwise might not. In addition, the methods and systems may help lower cost and improve timely availability of CTS treatments. Overall, through these and other effects, the methods and systems of the present disclosure may decrease morbidity associated with CTS or other hand/wrist conditions.

The methods and systems of the present disclosure are particularly well-suited for use by a patient in a non-medical setting, such as in the patient's home. The patient may, for example, access a web site that displays questions and instructions for self-assessment of CTS-related symptoms, collects patient's responses, calculates a non-diagnostic CTS assessment, and then, based on the CTS assessment, makes a recommendation to the patient, such as whether to seek further medical care for CTS or how urgently to seek care, based on those responses.

As used herein, the terms “diagnosis” and “diagnostic” refers to a medical conclusion by a trained and licensed medical professional, such as a physician with CTS expertise.

A used herein, the terms “assessment” and “assess” refer to an evaluation that is not a diagnosis. In particular, the terms include evaluations by non-medical professionals, such as patients. CTS assessments obtained using the methods and systems described herein are designed to have a high correlation with CTS diagnoses, but such assessments are not made by an appropriately-trained physician, and are, therefore, not diagnoses. CTS assessments described herein are designed to assist patients in determining whether or how to obtain a CTS diagnosis.

As used herein, the term “guideline” refers to an instruction that is recorded in a visual or oral medium, and that is not an instruction provided in real time by a medical professional able to make a CTS diagnosis. Guidelines do no include modified and specific instructions of the type given by a physician to a patient during a real time examination by a physician. Guidelines, in some embodiments, may be provided via writing, diagrams, audio recordings, video recordings, and any combinations thereof. In some embodiments, guidelines are provided in writing. In more particular embodiments, the guidelines provided in writing may be accompanied by an audio recording or the written guidelines being read aloud. In some embodiments, guidelines are provided as an audio recording. In more particular embodiments, guidelines provided in writing or as an audio recording may be accompanied by links to additional information about the guidelines, which may include further writing, diagrams, audio recordings, video recordings, and any combinations thereof. The term “guideline” as used herein refers to instructions in accordance with embodiments of the present disclosure, and not to other instructions or recommendations available to medical professionals, such as “clinical practice guidelines” (CPGs) of the type provide by orthopedic and other medical professional societies (unless such guidelines are revised in future to encompass guidelines according to the present disclosure).

An individual guideline for a CTS symptom refers a single instruction or to a collection of instructions that, when performed by the patient, provide information regarding whether the patient exhibits the CTS symptom.

As used herein, “self-assessed” and “self-assessment” as used herein refer to the performance of an evaluation according to guidelines without the assistance of a medical profession competent to diagnose CTS. In some embodiments, the patient may complete self-assessed steps on their own, without requiring any other real time human assistance. In some embodiments, the patient may be assisted in real time by another person who is not a medical professional, such as a friend, family member, or non-medically licensed care giver. In some embodiments, the patient may be assisted in real time by a medical profession who is, nevertheless, not competent of provide a CTS diagnosis, such as professionally-licensed care giver, nurse, nurse practitioner, or non-CTS expert physician. In some embodiments, the patient may be assisted in real time by a non-physician medical professional, particularly one who is not otherwise competent to provide a CTS diagnosis.

Systems according to the present disclosure are discussed in further detail below. However, in some embodiments, the methods may be performed using a computer, such as an electronic device containing a processor, a memory, a display, and an input. The electronic device may, for example, be a mobile phone or smartphone, a general use tablet, a dedicated patient input tablet, or a laptop or desktop computer (PC). In some embodiments, the electronic device may be capable of communicating with other devices over a network, such as a mobile phone network or other wireless network, or a wired network connection. In particular embodiments, system may include an electronic device able to access a web page and a server able to host the web page and use it to send and receive information with the electronic device.

In particular, the disclosure provides a system for implementing such a method that involves i) a computer, such as a dedicated server or a server hosting a cloud-based system, that provides the assessment questions to a remotely-located patient and stores results, typically via a web page or app, and ii) a separate electronic device, such as a smartphone, tablet, PC, or other consumer electronic, that the patient uses to view or hear the questions and to enter responses and, typically, to also receive the results of the assessment. For example, a patient might access a web site via a smartphone or PC and send a request to perform the self-guided CTS assessment. The web site might then call upon servers that support the web site to run a program that provides the questions to the patient via the web site, stores the patients responses to the questions, and provides the patient with results after the self-assessment has been completed, typically also through the web site or potentially through an email, text, or other communication.

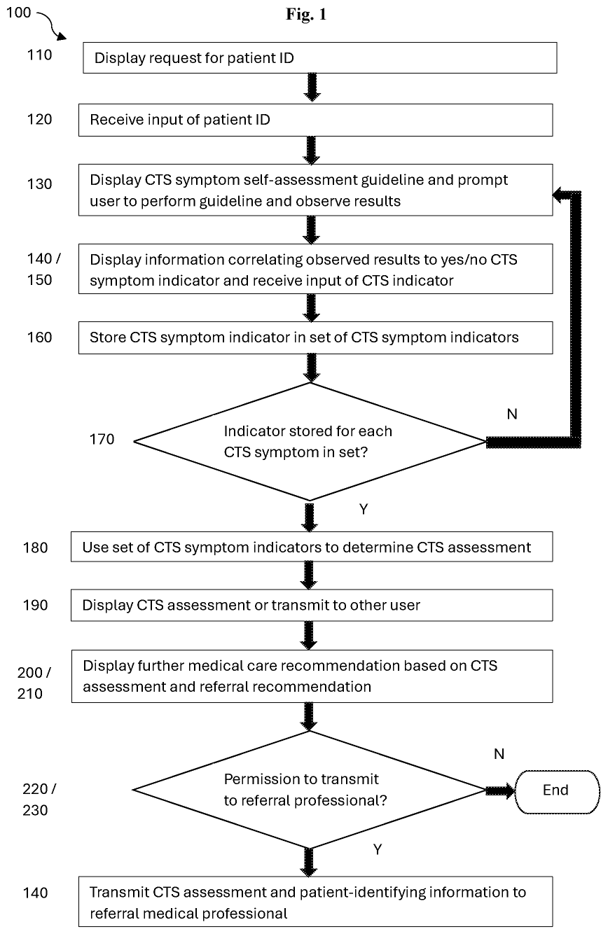

Referring now to FIG. 1, the disclosure provides a computer-implemented method 100 for guideline-directed self-assessment of CTS using a set of self-assessed CTS symptoms.

In step 110, a request for a patient ID associated with a patient is displayed. In some embodiments, the request may include steps to create a patient ID, which may then be stored in the memory of an electronic device. In some embodiments, the patient ID may be associated with additional patient-associated information, such as name, contact information, or other patient records, and any combinations thereof. In some embodiments, the patient ID may have been previously provided to the patient by a medical professional.

In step 120, the patient ID is input, typically by the patient. In some embodiments, the patient ID is also validated.

Some embodiments, particularly those that allow anonymous CTS assessment, steps 110 and 120 may be omitted.

In step 130, a first CTS symptom self-assessment guideline is displayed and the patient is prompted to perform the guideline. In some embodiments, the guideline may include instructions for observing results of performing other instructions in the guideline. In some embodiments, the self-assessment guidelines may be a yes/no question. In some embodiments, the guideline may instruct the patient to perform a physical evaluation. In some embodiments, the guideline may instruct the patient to answer a question regarding current or recent physical state without the need for a physical evaluation.

In step 140, information is displayed regarding how results observed by the patient upon performing the guideline in step 130 correlate with whether the patient does or does not exhibit the first CTS symptom, and the patient is prompted to input a yes/no indicator for the CTS symptom. A yes/no indicator need only allow the patient to express that, as determined according to the guideline, they do exhibit the CTS symptom by inputting a “yes” indicator, and they do not exhibit the CTS symptom by inputting the “no” indicator. In some embodiments, the yes/no indicator may include the words “yes” or “no”. In other embodiments, the yes/no indicator may include other words, such as “positive” or “negative,” “present” or “not present” or “absent,” etc. In some embodiments, the yes/no indicator may include a non-verbal visual indicator, such as check or X. In some embodiments, the yes/no indicator may include an auditory indicator, such as spoken words.

In step 150, the patient inputs a yes/no indicator for the first CTS symptom based on the information provided in step 140. In some embodiments, the patient may only be allowed to select one of the yes/no indicators to avoid errors in the self-assessment. In some embodiments, the patient may select the appropriate yes/no indicator on a visual display. In some embodiments, particularly those in which the yes/no indicator is an auditory indicator, the patient may provide an oral response of the yes/no indicator.

In step 160, the input yes/no indicator for the first CTS symptom is stored, typically in connection with the patient ID, as part of a set of CTS symptom indicators.

In step 170, the method determines if CTS symptom indicators have been stored for each symptom in the set of self-assessed CTS symptoms. If yes, then the method proceeds to step 180. If not, then the method returns to step 130 and repeats steps 130-160 with respect to a second or subsequent CTS symptom in place of the first CTS symptom.

In some embodiments, the set of CTS symptoms includes between three and six symptoms, or between three and five CTS symptoms. In specific embodiments, the set of CTS symptoms includes three, five, or six CTS symptoms. In some embodiments, the CTS symptoms are selected from the following: S1) numbness in median nerve distribution; S2) subjective night symptoms; S3) thenar atrophy; S4) phalen test; S5) loss of 2-point discrimination; and S6) tinel sign.

In a some embodiments, the set of CTS symptoms includes S1-S6. In some embodiments, the set of CTS symptoms includes S1-S5. In some embodiments, the set of CTS symptoms includes S1, S2 and S4.

In step 180, the stored set of CTS symptom indicators is used to determine a CTS assessment for the patient. In some embodiments, the CTS assessment may be a yes/no assessment, indicating whether the patient is assessed as having CTS or a not having CTS, respectively. In some embodiments, the CTS assessment may be a graduated assessment, such as a yes/likely/no assessment, indicating whether the patient has a certain high likelihood of having CTS (e.g. >90% chance), a certain low likelihood of having CTS (e.g. a ≤10% chance), or an intermediate likelihood of having CTS (e.g. a >10%, but ≤90% chance).

In some embodiments, this CTS assessment may be determined according to any of the methods provided in the Examples below. In some embodiments, a sum calculated for the set of CTS indicators, in which a yes indicator is assigned 1 and a no indicator is assigned 0 may be used to determine the CTS assessment by comparing the sum to a cutoff point. In some embodiments, the sum may be a simple sum of the numeric values of the CTS indicators. In other embodiments, the sum may be a weighted sum, in which the CTS indicators are multiplied by constants selected to reflect the relative values of the CTS indicators in determining an accurate CTS assessment. In other embodiments, more complex calculations with the CTS indicators may be performed, some of which are described in more detail in the Examples.

In some embodiments, the cutoff point is a number selected to provide a desired accuracy level for CTS assessments performed according to method 100. In embodiments that provide a yes/no CTS assessment, there may be a single cutoff point demarcating the boundary between yes and no. In embodiments that provide a graduated CTS assessment, multiple cutoff points may be provided, with one cutoff point demarcating each boundary between graduated CTS assessment categories.

In some embodiments, when the CTS assessment obtained using this method is compared to an in-clinic CTS diagnosis by a physician with CTS expertise the CTS assessment may have a selected accuracy level or a combination of selected accuracy levels. Accuracy levels may be reflected in specificity, sensitivity, positive predictive value, negative predictive value, ROC, or any combinations thereof. For accuracy levels not normally expressed as percentages, these percentages may be adjusted to numeric values (e.g. 90%=0.9).

In a some embodiments, the set of CTS symptoms includes S1-S6 and the CTS assessment is a yes/no assessment. In some embodiments, the set of CTS symptoms includes S1-S5 and the CTS assessment is a yes/no assessment. In some embodiments, the set of CTS symptoms includes S1, S2 and S4 and the CTS assessment is a yes/no assessment. In these embodiments, when the CTS assessment obtained using this method is compared to an in-clinic CTS diagnosis by a physician with CTS expertise: accuracy may be at least 75%, at least 80%, or in a range between 75% and 99%, 75% and 95%, 75% and 90%, 75% and 85%, 80% and 99%, 80% and 95%, 80% and 90% or 80% and 85%; Sn/Sp may be at least 0.85, at least 0.9, or in a range between 0.85 and 0.99, 0.85 and 0.95, 0.85 and 0.9, 0.9 and 0.99, or 0.9 and 0.95; J value may be at least 0.6, at least 0.65, at least 0.7 or at least 0.75, or in a range between 0.6 and 0.85, 0.6 and 0.8, 0.6 and 0.75, 0.65 and 0.85, 0.65 and 0.8, 0.65 and 0.75, 0.7 and 0.85, 0.7 and 0.8, 0.75 and 0.85, or 0.75 and 0.8.

In step 190, the CTS assessment is displayed to the patient. In some embodiments, the CTS assessment may be displayed via writing, diagrams, audio recordings, video recordings, and any combinations thereof. In some embodiments, the CTS assessment is provided in writing. In more particular embodiments, the CTS assessment provided in writing may be accompanied by an audio recording or the written assessment being read aloud. In some embodiments, the CTS assessment is provided as an audio recording. In more particular embodiments, CTS assessments provided in writing or as an audio recording may be accompanied by links to additional information about the CTS assessment, which may include further writing, diagrams, audio recordings, video recordings, and any combinations thereof.

In some embodiments, rather than display the CTS assessment to the patient, the CTS assessment is instead provided to a medical professional, such as a medical professional associated with the patient ID.

In some embodiments method 100 ceases after step 190. However, in other embodiments, further steps 200-240 are performed to assist the patient in acting upon the CTS assessment.

In step 200, a further medical care recommendation based upon the CTS assessment is displayed. In some embodiments, the CTS further medical care recommendation may further take into account an applicable standard of care for CTS. In some embodiments, the CTS further medical care recommendation may also be based on other patient-associated information.

In some embodiments, for a CTS assessment of “no,” the further medical care recommendation may indicate that further care for CTS is likely not needed. In some embodiments, for CTS assessment of “no,” the further medical care recommendation may indicate that further medical care for other hand/wrist conditions may be needed or should be considered.

In some embodiments, for a CTS assessment of “yes” or another indicator of a high likelihood of the patient having CTS, the further medical care recommendation may be to promptly seek professional medical care. In particular embodiments, the further medical care recommendation may be to seek professional medical care from a CTS expert, such as a surgeon.

In some embodiments with graduated CTS assessments, the further medical care recommendation for an intermediate CTS assessment may be to seek professional medical care, particularly if certain other criteria are met, such as the presence of other symptoms consistent with CTS or the presence of other risk factors in the patient's medical history. In some embodiments, the further medical care recommendation may be to repeat the guideline-directed self-administered CTS assessment after a specified period of time, such a one, three, or six months, or one year.

In some embodiments in which the CTS assessment is “no” or an intermediate CTS assessment, the further medical care recommendation may include to seek medical care for other hand/wrist conditions consistent with the patient's symptoms.

In step 210, if the further medical care recommendation is to seek professional medical care, at least one referral recommendation may be displayed, typically based on a patient-provided location. In some embodiments, the patient-provided location may be obtained by a patient-location feature of the electronic device. In some embodiments, the location may be provided from patient-associated information. In some embodiments, the location may be provided by the patient in response to a prompt requesting the patient's location, or a location where the patient would like to receive medical care.

In some embodiments, the referral recommendation may include medical professionals specifically trained in CTS assessment or treatment.

In some embodiments, the referral recommendation may include a recommendation to seek expedited treatment or further diagnosis. Such a referral recommendation may be provided when, for example, the CTS assessment indicates a particularly high likelihood that the patient has CTS or that the patient likely has particularly severe CTS. A particularly high total score in the CTS assessment, or a particularly high score for one or more specific questions in the CTS assessment may indicate either of these factors.

In step 220, a request to transmit the CTS assessment and, optionally, patient-associated information including at least the patient name and contact information, to a medical professional is displayed. In some embodiments, medical professional may be one associated with the patient ID. In some embodiments, the medical professional may be one from the referral recommendation of step 210. In some embodiments, the request may be to transmit the CTS assessment to multiple medical professionals. In some such embodiments, the method may include a separate request for each such medical professional.

In step 230, the patient grants or denies the request to transmit the CTS assessment. In some embodiments, this step may include selecting at least one medical profession from the referral recommendation of step 210.

If the request is denied, the method ends, or continues to further medical-related steps, such as providing links to further information on CTS, other hand/wrist conditions, information regarding referrals or appropriate medical specialties, or other information not described in detail here.

If the request is granted, then, in step 240, the CTS assessment and, optionally, patient-associated information, is transmitted to the medical professional.

In an alternative embodiment, if, in step 200, the further medical care recommendation is to seek professional medical care, the CTS assessment may be automatically transmitted to a medical professional associated with the patient ID, with no further permissions or input from the patient being required.

In some embodiments, the method includes transmission of information to and from a patient electronic device and a patient data processing center.

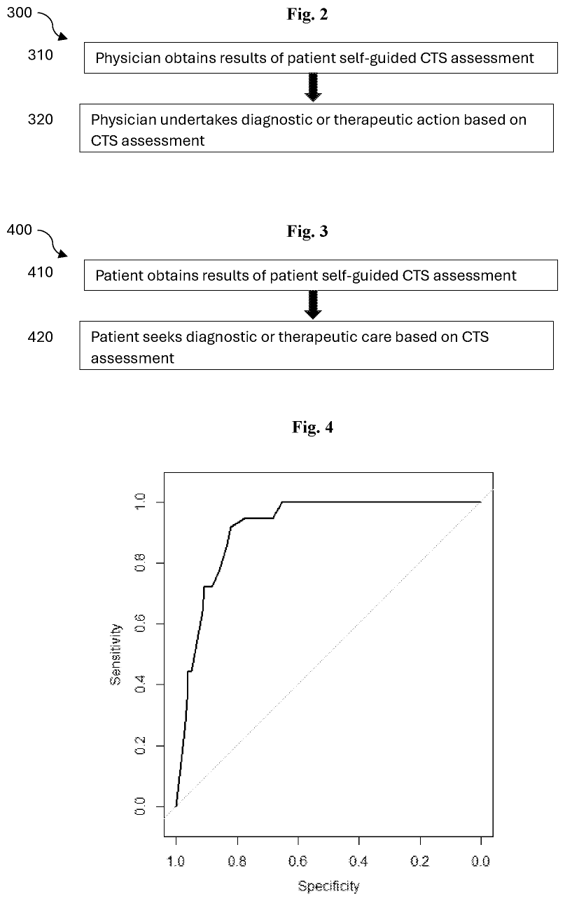

FIG. 2 illustrates a method 300 for diagnosis of CTS in a patient by a physician. In step 310, the medical professional obtains results of a patient self-guided CTS assessment for a patient, where the CTS assessment was determined using method 100. In some embodiments, the medical professional may be a physician, particularly a physician with CTS expertise.

In step 320, based on the CTS assessment, the medical professional undertakes a diagnostic or therapeutic action. In some embodiments, the diagnostic or therapeutic action may be in accordance with a standard of care. In some embodiments, the diagnostic or therapeutic action may otherwise be taken at the discretion of the medical profession. In more specific embodiments, the medical professional may take into account other patient-specific factors along with the CTS assessment in determining diagnostic or therapeutics action. Such other patient-specific factors may include the patient's age, co-morbidities, particularly any orthopedic co-morbidities, co-morbidities affecting the risk associated with surgery, co-morbidities affecting inflammatory responses, and co-morbidities affecting healing, such as any tendencies to develop fibrosis or diabetes, and the patient's lifestyle, such as occupation or other regularly necessary tasks that require fine motor control in or repetitive motion of the affected hand/wrist or arm. These and other factors may be considered by the medial profession while exercising discretion to determine whether the patient is sufficiently likely to benefit from, and not be subject to unduly high risk resulting from, any potential diagnostic or therapeutic measures, such as surgery, or non-surgical interventions and treatments.

In some embodiments, in step 310 the results contain a recommendation for expedited treatment, in which case the medical professional may proceed to step 320 more quickly than is typical for that medical professional when evaluating or treating a similar patient with suspected or diagnosed CTS.

In some embodiments, the diagnostic action may simply be a physical examination of the patient by the medical professional. In some embodiments, the diagnostic action may be assessment of the CTS symptoms by or under direction of the medical professional. In some embodiments, the diagnostic action may include diagnostic testing, such as UT or EDS.

In some embodiments, the therapeutic action may include surgery to alleviate CTS or referral of the patient to another physician with expertise in CVS, such as a surgeon with expertise in CTS. In some embodiments, in determining the therapeutic action, the medical professional may choose to use the CTS assessment as if it were a CTS diagnosis.

FIG. 3 illustrates a method 400 for a patient to seek CTS diagnosis or treatment. In step 410, the patient performs a method 100 and obtains results of a patient self-guided CTS assessment. In some embodiments, the results may include an indication that the patient should seek expedited treatment or further diagnosis of CTS.

In step 420, based upon the CTS assessment, the patient determines whether to seek further medical care. If the patient determines to not seek further medical care, the method ends. If the patient determines to seek further medical care, then the method proceeds to step 430.

In step 430, the patient, upon seeking further medical care, obtains a diagnostic or therapeutic recommendation and acts upon such recommendation. In some embodiments, the further medical care is provided by a medical professional according to method 300, particularly in accordance with a standard or care on in the discretion of the medical professional. In some embodiments, the patient provides the CTS assessment to the medical professional from whom care is sought. In some embodiments, the patient undergoes recommended diagnostic testing, such as that described in connection with step 320 above. In some embodiments, the patient undergoes recommended therapeutic care, such as that described in connection with step 320 above, with does or does not seek professional medical care. If the patient does seek professional medical care, this may be based on a referral recommendation provided in step 220 of method 100. In some embodiments, the professional medical care may include a diagnostic action.

### Systems

The disclosure further provides systems for self-administered CTS assessment. In particular embodiments, such a system is able to perform the steps of method 100. In some embodiments, the system is able to perform steps 130-190 of method 100.

In some embodiments, the system includes an electronic device containing a processor, memory, display, input, and I/O interface capable of storing instructions within the memory that, when executed by the processor, cause the electronic device to perform or communicate via the I/O interface over a network with a patient data processing center that includes a processor, a memory and an I/O interface, and cause the patient data processing center to perform a self-administered CTS assessment method of the present disclosure, such as method 100, or steps 130-190 of method 100.

In some embodiments, the system includes a patient data processing center that includes a processor, a memory, and an I/O interface capable of storing instructions within the memory that, when executed by the processor cause the patient data processing center to perform or communicate via the I/O interface over a network with an electronic device containing a processor, memory, display, and input and cause the electronic device to perform a self-administered CTS assessment method of the present disclosure, such as method 100, or steps 130-190 of method 100.

The functionality described herein for a system can be implemented either on dedicated hardware, as a software instance running on dedicated hardware, or as a virtualized function instantiated on an appropriate platform, e.g., a cloud infrastructure. In some embodiments, such functionality may be completely software-based and designed as cloud-native, meaning that they are agnostic to the underlying cloud infrastructure, allowing higher deployment agility and flexibility.

The network may include any appropriate wired or wireless data transmission, and may use any suitable communications protocol. For example, the network may provide communication may be serial or parallel, through Universal Serial Bus (USB) (wired or wireless), Ethernet, Bluetooth, Near Field Communications (NFC), radio frequency (RF), infrared, or WiFi, and any combinations thereof.

In some embodiments, the patient ID, patient-associated information, guidelines, CTS symptom indicators, instructions for calculating a CTS assessment using the CTS symptom indicators, and CTS assessment may be stored in the memory of the patient data processing center.

In some embodiments, any information displayed may be displayed by and any inputs may be obtained using the electronic device.

### EXAMPLES

### Example 1—Evaluation of Web-Based CTS Self-Assessment Method

Accuracy levels of a web-based CTS self-assessment method for determining whether a patient has CTS was assessed. The results of a CTS-6 method obtained in clinic (in-clinic CTS diagnosis) via patient evaluation by a medical professional were used as the reference standard. Sensitivity (Sn), specificity (Sp), positive predictive value (PPV) and negative predictive value (NPV) were calculated.

Goals of this example were:
Goal 1—To determined accuracy, as reflected in Sn, Sp, PPV, and NPV, for a web-based CTS self-assessment method that provides instructions to patients for self-assessment of CTS symptoms, as compared to the reference standard.
Goal 2—To determine accuracy, as reflected in Sn, Sp, PPV, and NPV, of the web-based CTS self-assessment method for the individual CTS symptoms (6 different symptoms, reflected in the designation CTS-6), compared to the reference standard.
Goal 3—To create an accurate, novel scale for predicting CTS, using all CTS-6 symptoms, or a subset of CTS-6 symptoms.

The CTS-6 symptoms were as follows: S1) numbness in median nerve distribution; S2) subjective night symptoms; S3) thenar atrophy; S4) phalen test; S5) loss of 2-point discrimination; and S6) tinel sign.

### Methods

This example was conducted using a sample of 198 patients. 36 patients, representing 18.2% of the total, had been diagnosed as having CTS using in-clinic CTS diagnosis.

### Inclusion & Exclusion Criteria

This was a single-center study in an academic upper-extremity surgery division. The center was a tertiary referral center and serves a predominantly rural patient population in the northeastern United States. Two surgeons, both of whom are fellowship trained in hand and upper-extremity surgery, participated in this investigation. Clinic visits were conducted at three separate clinic sites, all of which are part of the healthcare system.

Inclusion criteria for screening included all patients ≥18 years of age presenting with any atraumatic complaints or assessments involving the upper limb. Patients who had undergone an upper-extremity surgical procedure within six weeks of their scheduled outpatient visit were excluded. Patients requiring the use of language translation services or a sign-language interpreter were not eligible for screening. Patients who failed to complete the entire internet-based questionnaire or had their in-person examination results incompletely documented were excluded.

### Patient Screening and Remote Examination:

After the check-in process for outpatient visits, eligible patients were approached by one of two post-graduate research fellows. The research fellows provided patients with a detailed study sheet and verbally described the investigation. Patients were informed that study participation was voluntary and that they would not receive monetary compensation for their participation.

After patients consented to participate, they were provided with a QR code that directed them to a website. Eligible patients were allowed to use one of two devices: a personal smartphone with internet access or a clinic-owned tablet. Patients completed the questions and self-assessed CTS symptoms according to guidelines on the device prior to their consultation with the attending surgeon. The webpage contained a brief description of CTS as well as a series of basic demographic questions and questions related to their peripheral nerve symptoms. Next, patients completed the six history and examination items contained in the CTS-6, which were modified for self-administration (without surgeon guidance) based on a prior investigation of the CTS-6 in the telemedicine setting (Grandizio L C, Barreto-Rocha D, Foster B K, Udoeyo I F. Evaluation of a comprehensive telemedicine pathway for carpal tunnel syndrome: a comparison of virtual and in-person assessments. J Hand Surg Am. 2022; 47(2): 111-119), as well as a self-administered a Durkan compression test. All survey responses were stored anonymously on a secure, cloud-based, HIPAA-complaint server. Patients were individually assigned study ID numbers.

### In-Clinic Diagnosis

Following the self-administered internet assessment, patients were seen in-clinic by their attending hand surgeon. Both surgeons were blinded to the results of the self-administered responses from the internet survey. During the clinic visit, the surgeon administered the CTS-6 evaluation.

Diagnostic performance metrics were calculated using the following formulas:

Sensitivity=true positives/(true positives+false negatives)

Specificity=true negatives/(true negatives+false positives)

Positive predictive value=true positives/(true positives+false positives)

Negative predictive value=true negatives/(true negatives+false negatives)

Accuracy/agreement=((true positives+true negatives)/total number of patients)*100

In these formulas:
True positives refer to the number of patients who received a positive/non-zero value in both the web-based CTS self-assessment method and the in-clinic diagnosis for CTS or the CTS symptom
True negatives refer to the number of patients who received a negative/zero value on both the web-based CTS self-assessment method and the in-clinic diagnosis for CTS or the CTS symptom
False positives refer to the number of patients who received a positive/non-zero value on the web-based CTS self-assessment method, but a negative/zero value on the in-clinic CTS diagnosis for CTS or the CTS symptom
False negatives refer to the number of patients who received a negative/zero value on the web-based CTS self-assessment method, but a positive/non-zero value on the in-clinic CTS diagnosis for CTS or the CTS symptom.

All of these metrics were expressed as proportions except accuracy. Accuracy was expressed as a percentage. Correlations between web-based and in-clinic evaluations were either phi coefficients for binary variables (all except CTS-6 total scores) or Pearson's correlation for the correlation between CTS-6 total scores.

Binary logistic regression was used to develop test models for predicting in-clinic CTS diagnosis using the web-based CTS self-assessment method version symptoms/data. For this procedure, the sample was randomly divided into 2 parts. One part consisted of 70% of participants and this part was used to fit the models to create prediction models. The second part contained the remaining 30% of participants and it was used as a crossvalidation sample to test the prediction model on a sample it was not fitted on. Before doing binary logistic regression, all web-based CTS self-assessment were recoded to 1 and 0 from CTS weights.

Based on the results of these regression models, scores were created from predictors in the models in 2 ways—as predicted probabilities calculated using the logistic function:

Predicted value=1/(1+2.718**−(const+B1*X1+B2*X2+B3*X3+ . . . +Bn*Xn)

where:
2.718 is the base of the natural logarithm e,
const is the constant from the model,
B1, B2 . . . are regression coefficients, and
X1, X2 . . . are predictors included in the model.

Optimal cut-off points and optimal scores were selected using the Youden's J statistic, calculated as: Sensitivity+Specificity−1. They were calculated in R v.4.4.0, and using the pROC package.

### Goal 1 Results

First, the ability of the web-based self-assessments for each CTS symptom to predict the in-clinic CTS diagnosis was calculated by crosstabulating self-assessment against the in-clinic CTS diagnosis. Results are presented in Table 1.

TABLE 1
Sensitivity, specificity, positive predictive value and negative predictive value for individual
symptoms of the web-based CTS self-assessment method used to predict the in-clinic CTS diagnosis

| | | Positive Predictive | | Correlation | |
|---|---|---|---|---|---|
| | Sensitivity / | Value / Negative | | (Phi or | Youden's |
| SYMPTOM | Specificity | Predictive Value | Agreement | Pearson's r) | J statistic |
| CTS-6 COMPONENTS | | | | | |
| S1. Numbness in median nerve | 0.89/0.69 | 0.39/0.97 | 73% | 0.45 | 0.58 |
| distribution | | | | | |
| S2. Subjective night symptoms | 0.83/0.77 | 0.45/0.95 | 78% | 0.49 | 0.60 |
| S3. Thenar atrophy | 0.47/0.85 | 0.40/0.88 | 78% | 0.30 | 0.32 |
| S4. Phalen test | 0.78/0.82 | 0.49/0.94 | 81% | 0.51 | 0.60 |
| S5. Loss of 2-point | 0.81/0.68 | 0.36/0.94 | 70% | 0.38 | 0.48 |
| discrimination | | | | | |
| S6. Tinel sign | 0.83/0.77 | 0.51/0.89 | 82% | 0.40 | 0.60 |
| CTS-6 Total Score | N/A | N/A | N/A | 0.61 | |
| OTHER EXAM ITEMS | | | | | |
| Durkan Compression test | 0.69/0.85 | 0.51/0.93 | 82% | 0.49 | 0.55 |
| OVERALL CTS ASSESSMENT | | | | | |
| CTS Assessment (WEB-BASED | 0.92/0.90 | 0.53/0.98 | 84% | 0.61 | 0.81 |
| CTS SELF-ASSESSMENT | | | | | |
| METHOD) | | | | | |

All correlations were statistically significant. The correlation between the CTS-6 total score obtained using the web-based CTS self-assessment method and the in-clinic CTS diagnosis was 0.61, which is somewhat lower than the correlation of 0.71 between the probability of CTS and the assessment given by a panel of clinicians reported in the paper on the creation of CTS-6 (Graham, B., Regehr, G., Naglie, G., & Wright, J. G. (2006). Development and Validation of Diagnostic Criteria for Carpal Tunnel Syndrome. The Journal of Hand Surgery, 31 (6), 919.e1-919.e7. doi.org/10.1016/j.jhsa.2006.03.005).

The web-based CTS self-assessment method, for all symptoms except S3, was able to correctly identify more than half of in-clinic diagnosed CTS cases. The web-based CTS self-assessment method had high sensitivity and specificity, however, only around a half of cases assessed as having CTS using the web-based CTS self-assessment method were also diagnosed with CTS by the in-clinic CTS diagnosis (PPV).

### Goal 2 Results

Web-based CTS self-assessment method CTS symptom-specific results, Durkan pressure test outcome and CTS assessment were crosstabulated with their in-clinic counterparts and the abilities of the web-based CTS self-assessment method to predict in-clinic evaluation for each CTS symptom, the Durkan pressure test, and CTS diagnosis were calculated. Results are presented in Table 2.

TABLE 2
Sensitivity, specificity, positive predictive value and negative predictive value for individual symptoms
assessed using the web-based CTS self-assessment method compared to in-clinic CTS diagnosis

| | | Positive Predictive | | Correlation | |
|---|---|---|---|---|---|
| | Sensitivity / | Value / Negative | | (Phi or | Youden's |
| SYMPTOM | Specificity | Predictive Value | Agreement | Pearson's r) | J statistic |
| CTS-6 COMPONENTS | | | | | |
| S1. Numbness in median nerve | 0.86/0.74 | 0.52/0.94 | 77% | 0.53 | 0.60 |
| distribution | | | | | |
| S2. Subjective night symptoms | 0.82/0.80 | 0.54/0.94 | 80%/ | 0.54 | 0.62 |
| S3. Thenar atrophy | 0.75/0.84 | 0.29/0.97 | 83% | 0.39 | 0.59 |
| S4. Phalen test | 0.79/0.85 | 0.60/0.94 | 84% | 0.59 | 0.64 |
| S5. Loss of 2-point | 0.61/0.69 | 0.51/0.78 | 67% | 0.30 | 0.31 |
| discrimination | | | | | |
| S6. Tinel sign | 0.82/0.80 | 0.49/0.96 | 88% | 0.53 | 0.62 |
| CTS-6 Total Score | N/A | N/A | N/A | 0.69 | |
| OTHER EXAM ITEMS | | | | | |
| Durkan Compression test | 0.77/0.87 | 0.53/0.98 | 84% | 0.56 | 0.64 |
| OVERALL CTS ASSESSMENT | | | | | |
| CTS Assessment (WEB- | 0.92/0.90 | 0.53/0.98 | 84% | 0.61 | 0.81 |
| BASED CTS SELF- | | | | | |
| ASSESSMENT METHOD) | | | | | |

All presented correlations are statistically significant. Numbers in the table are symptoms of how well, for each symptom, the web-based CTS self-assessment method predicted in-clinic CTS diagnosis.

Results show that all correlations between web-based CTS self-assessments and their in-clinic counterparts were statistically significant. Additionally, the accuracy of all web-based CTS self-assessments in predicting the values of their in-clinic counterparts is better than chance. Using the web-based CTS self-assessment method, S5 had a positive value for only 61% of cases that received positive scores on the in-clinic CTS diagnosis for this symptom.

Overall, the web-based CTS self-assessment method was much better at predicting negative values of in-clinic diagnoses.

### Goal 3 Results

Next, descriptive statistics for total scores obtained using the web-based CTS self-assessment method and in-clinic CTS diagnosis were calculated and are presented in Table 3.

TABLE 3
Descriptive statistics of web-based CTS self-assessment
method and in-clinic CTS diagnosis total scores

| | Mean | SD | Skewness | Kurtosis |
|---|---|---|---|---|
| Web-based CTS-6 tool Total score | 7.85 | 8.08 | 0.749 | −0.551 |
| In-clinic CTS diagnosis Total score | 5.25 | 6.7 | 1.329 | 0.942 |

Results show that the CTS-6 total score derived from the web-based CTS self-assessment method had a higher score than its in-clinic equivalent by approximately 2.6, and somewhat higher variability. CTS-6 total scores obtained using both evaluation methods were positively skewed, but the in-clinic CTS diagnosis had a stronger skewness, which is to a large extent due to the fact that there more participants scored 0 on the in-clinic CTS diagnosis (46%) than on the web-based CTS self-assessment method (36%).

If the in-clinic results are treated as a reference, this indicates that the web-based CTS self-assessment methods might be overestimating the total CTS-6 scores. To check whether this difference is general or is created by specific items, percentages of positive values for all CTS-6 symptoms were calculated and compared between the web-based CTS self-assessment method and the in-clinic CTS diagnosis. Results are presented in Table 4.

TABLE 4
Comparison of percentages of patients with positive scores using
in-clinic CTS diagnosis and web-based CTS self-assessment method

| | Web- | |
|---|---|---|
| | based | In-clinic |
| S1. Numbness in median nerve distribution | 41% | 25% |
| S2. Subjective night symptoms | 34% | 22% |
| S3. Thenar atrophy | 21% | 8% |
| S4. Phalen test | 29% | 22% |
| S5. Loss of 2-point discrimination | 41% | 34% |
| S6. Tinel sign | 18% | 12% |
| Durkan compression test | 25% | 18% |
| CTS assessment | 31% | 18% |

The results show that the web-based CTS self-assessment method produces more positive values for all CTS symptoms. The share of patients diagnosed with CTS using the web-based CTS self-assessment method is 1.7 times higher than based on the in-clinic CTS diagnosis. If the in-clinic CTS diagnosis is taken to be a valid reference, this indicates that the web-based CTS self-assessment method is too sensitive.

However, if the sensitivity of the web-based CTS self-assessment method was decreased so that it reported roughly a similar percentage of positive assessments as the in-clinic CTS diagnosis, by increasing the cut-off point to 16.5 (instead of 12), the overall balance of symptoms would decrease. If the sensitivity was decreased to compensate for the difference between means of the web-based CTS self-assessment method and in-clinic CTS diagnosis of CTS scores, the overall balance of sensitivity and specificity would also decrease. These effects are calculated in Table 5.

TABLE 5
Diagnostic statistics and J scores for different cut-off points for web-based
CTS self-assessment method total scores for establishing the assessment of CTS

| | | | | | | Correlation with the | |
|---|---|---|---|---|---|---|---|
| | | | | | | clinical response | |
| | Sensitivity | Specificity | PPV | NPV | Accuracy | (Phi or Pearson r) | J |
| Cut-off point is 16.5 | 0.64 | 0.91 | 0.62 | 0.92 | 0.86 | 0.53 | 0.55 |
| Cut-off point is 14.6 | 0.72 | 0.90 | 0.62 | 0.94 | 0.87 | 0.53 | 0.62 |
| Cut-off point is 12 | 0.92 | 0.90 | 0.53 | 0.98 | 0.84 | 0.61 | 0.81 |

Inspection of the ROC curve created by using the web-based CTS self-assessment method CTS-6 score as a predictor and the in-clinic diagnosis as a binary outcome (FIG. 4) indicated that the optimal total score cut-off point for determining if the patient does or does not have CTS is still around the default cut-off point of 12. Youlden's method for determining the optimal cut-off point indicated that 12.25 is the optimal cut-off point.

This indicates that 12 is the optimal cut-off point for determining if a patient does or does not have CTS using CTS-6 total score if the web-based CTS self-assessment method were used in the form presented above.

Next, a series of binary logistic regression models were fitted to examine if it was possible to create a shorter set of CTS symptoms that, when used in a web-based CTS self-assessment method, would predict the in-clinic CTS diagnosis as well or better than the web-based CTS self-assessment method using CTS-6.

To do that, the sample was randomly divided into 2 parts. 70% of patients were the part of the sample used to fit the regression models, while the remaining 30% were kept as a crossvalidation samples to test the models on a part of the sample it was not created from.

The following models were tested:
The full web-based CTS self-assessment method with all 6 CTS symptoms and the Durkan compression test as predictors
The full web-based CTS self-assessment method with the 6 CTS symptoms only
The web-based CTS self-assessment method using 5 symptoms from the above results that that were statistically significant, or the closest to being statistically significant
The web-based CTS self-assessment method using only the 3 statistically significant symptoms from the above results from model 1.

Summaries and coefficients of these variant web-based CTS self-assessment methods are presented in the following Tables 6-11.

First, a web-based CTS self-assessment method with all six CTS symptoms and further refinement using the Durkan compression test was created and assessed. Results are presented in Table 6.

TABLE 6
Binary logistic regression predicting in-clinic CTS diagnosis
based on web-based CTS self-assessment method using
all 6 CTS symptoms + Durkan compression test

| | | | Hosmer- | |
|---|---|---|---|---|
| | Nagelkerke | | Lemeshow | |
| Cox & Snell R Square | R Square | | test sig. | |
| | 0.587 | | 0.503 | |
| 0.361 | B | S.E. | Sig. | Exp(B) |
| S1. Numbness in median | 1.344 | 0.718 | 0.061 | 3.835 |
| nerve distribution | | | | |
| S2. Subjective night | 1.732 | 0.625 | 0.006 | 5.650 |
| symptoms | | | | |
| S3. Thenar atrophy | 0.978 | 0.656 | 0.136 | 2.659 |
| S4. Phalen test | 1.585 | 0.755 | 0.036 | 4.878 |
| S5. Loss of 2-point | 0.920 | 0.673 | 0.171 | 2.510 |
| discrimination | | | | |
| S6. Tinel sign | 0.466 | 0.691 | 0.500 | 1.594 |
| Web Durkan compression | 0.352 | 0.710 | 0.620 | 1.423 |
| test | | | | |
| Constant | −5.235 | 0.974 | p < 0.001 | 0.005 |

Fitted on 70% of sample. Statstically significant predictors are in bold.

The Hosmer-Lemeshow test indicates that the web-based CTS self-assessment method for CTS-6+Durkan compression test fits the data well. However, results for only two CTS symptoms were statistically significant, and one was close to the statistical significance threshold of 0.05. This indicated that the method could be simplified by excluding certain CTS symptoms without visible loss in predictive ability. Additionally, the results of the Durkan test was the least contributing predictor, having the lowest coefficient and being the least statistically significant. This indicated that the Drukan test could be first excluded from web-based CTS self-assessment method without substantially affecting its the accuracy and usefulness.

Next, a web-based CTS self-assessment method was created with just the CTS-6 symptoms. While this web-based CTS self-assessment method was essentially the same as the method including the Durkan test, upon which the CTS-6 total score was based, the CTS-6 total score in the method lacking the Durkan test was based on specific weights of symptoms and it is possible that reweighing symptoms might improve prediction. Results for the web-based CTS self-assessment method for CTS-6 only are ° presented in Table 7.

TABLE 7
Binary logistic regression predicting in-clinic
CTS diagnosis based on web-based CTS self-assessment
method using only all CTS-6 symptoms

| | | | Hosmer- | |
|---|---|---|---|---|
| | Nagelkerke | | Lemeshow | |
| Cox & Snell R Square | R Square | | test sig. | AUC |
| | 0.585 | | 0.349 | 0.92 |
| 0.360 | B | S.E. | Sig. | Exp(B) |
| S1. Numbness in median | 1.402 | 0.707 | 0.047 | 4.065 |
| nerve distribution | | | | |
| S2. Subjective night | 1.743 | 0.624 | 0.005 | 5.714 |
| symptoms | | | | |
| S3. Thenar atrophy | 1.029 | 0.647 | 0.111 | 2.799 |
| S4. Phalen test | 1.742 | 0.686 | 0.011 | 5.711 |
| S5. Loss of 2-point | 0.899 | 0.669 | 0.179 | 2.458 |
| discrimination | | | | |
| S6. Tinel sign | 0.579 | 0.648 | 0.371 | 1.785 |
| Constant | −5.249 | 0.973 | p < 0.001 | 0.005 |

Fitted on 70% of sample. Statstically significant predictors are in bold.

Fitted on 70% of sample. Statistically significant predictors are in bold.

Results of with the web-based CTS self-assessment method for CTS-6 only and lacking the Durkan test show that only the 3 of the 6 symptoms were statistically significant predictors of the in-clinic diagnosis. S6, tinel sign, was the weakest predictor, and also was not statistically significant. The overall prediction quality as indicated by R square measures was practically unaffected by removing the Durkan pressure test.

A web-based CTS self-assessment method that used only 5 CTS symptoms was tested. This method, which may be referred to as the CTS-5 method, lacked the S6 symptom, as it was the worst predictor of the set. Results for tests with the CTS-5 method are presented in Table 8.

TABLE 8
Binary logistic regression predicting in-clinic CTS diagnosis based
on web-based CTS self-assessment method using 5 CTS symptoms

| | | | Hosmer- | |
|---|---|---|---|---|
| | Nagelkerke | | Lemeshow | |
| Cox & Snell R Square | R Square | | test sig. | AUC |
| | 0.579 | | 0.344 | 0.92 |
| 0.357 | B | S.E. | Sig. | Exp(B) |
| S1. Numbness in median | 1.488 | 0.705 | 0.035 | 4.429 |
| nerve distribution | | | | |
| S2. Subjective night | 1.795 | 0.621 | 0.004 | 6.022 |
| symptoms | | | | |
| S3. Thenar atrophy | 0.977 | 0.636 | 0.125 | 2.656 |
| S4. Phalen test | 1.859 | 0.677 | 0.006 | 6.415 |
| S5. Loss of 2-point | 0.857 | 0.661 | 0.195 | 2.356 |
| discrimination | | | | |
| Constant | −5.204 | 0.959 | p < 0.001 | 0.005 |

Fitted on 70% of sample. Statstically significant predictors are in bold.

Fitted on 70% of sample. Statistically significant predictors are in bold.

In the web-based CTS self-assessment method using 5 CTS symptoms, the same 3 CTS symptoms were statistically significant predictors of in-clinic CTS diagnosis and the overall prediction ability of the method (as indicated by R square statistics) was practically the same as that of the web-based CTS self-assessment methods using 6 CTS symptoms.

Finally, a web-based CTS self-assessment method using only the 3 statistically significant CTS symptoms was assessed. Results are presented in Table 9.

TABLE 9
Binary logistic regression predicting in-clinic CTS diagnosis based
on web-based CTS self-assessment method using 3 CTS symptoms

| | | | Hosmer- | |
|---|---|---|---|---|
| | Nagelkerke | | Lemeshow | |
| Cox & Snell R Square | R Square | | test sig. | AUC |
| | 0.548 | | 0.610 | 0.904 |
| 0.338 | B | S.E. | Sig. | Exp(B) |
| S1. Numbness in median | 1.429 | 0.672 | 0.033 | 4.176 |
| nerve distribution | | | | |
| S2. Subjective night | 1.864 | 0.610 | 0.002 | 6.450 |
| symptoms | | | | |
| S4. Phalen test | 2.261 | 0.638 | p < 0.001 | 9.594 |
| Constant | −4.600 | 0.817 | p < 0.001 | 0.010 |

Fitted on 70% of sample. Statstically significant predictors are in bold.

Fitted on 70% of sample. Statistically significant predictors are in bold.

In this web-based CTS self-assessment method using 3 CTS symptoms, all predictors were statistically significant. This method was much shorter than the other web-based CTS self-assessment methods. However, its predictive accuracy was a slightly lower compared to the other web-based CTS self-assessment methods and it also had a slightly lower area under the ROC curve.

CTS scores were derived from the web-based CTS self-assessment methods using 6, 5, or 3 symptoms. In one assessment the CTS scores were derived as simple sums of involved predictors, where the non-zero values were assigned the value of 1. In another assessment, the CTS scores were derived as predicted probabilities created using the logistic function and model coefficients.

After this, ROC curves were created and areas under the curve calculated for each of these curves, and an optimal cutoff-point established using the Youden J statistic. Results are presented in Table 10.

TABLE 10
Optimal cut-off scores for predicting in-clinic
CTS diagnosis using scores derived from different
web-based CTS self-assessment methods

| | Optimal cutoff values |
|---|---|
| 6-symptom predicted probabilities | 0.109 |
| 6-symptom sum | 2.5 |
| 5-symptom predicted probabilities | 0.104 |
| 5-symptom sum | 2.5 |
| 3-symptom predicted probabilities | 0.15 |
| 3-symptom sum | 1.5 |

An overview of diagnostic characteristics of these CTS scores, when creating the predicted assessment using the optimal cutoff value, are given in Table 11, with the original web-based CTS-6 assessment given for comparison.

TABLE 11
Diagnostic characteristics of predictions of in-clinic CTS diagnosis,
based on the set of scores derived from the logistic analyses procedures
with optimal cut-off scores established using the Youden method

| | Sensitivity | Specificity | PPV | NPV | Accuracy | J |
|---|---|---|---|---|---|---|
| CTS-6 based assessment | | | | | | |
| Fitting sample (70%) | 0.96 | 0.80 | 0.52 | 0.99 | 0.83 | 0.76 |
| Testing sample (30%) | 0.78 | 0.88 | 0.58 | 0.95 | 0.87 | 0.66 |
| 70% of the sample (fitting sample) | | | | | | |
| 6-symptom predicted probabilities | 0.96 | 0.77 | 0.49 | 0.99 | 0.81 | 0.74 |
| 6-symptom simple sum | 0.96 | 0.76 | 0.48 | 0.99 | 0.80 | 0.73 |
| 5-symptom predicted probabilities | 1.00 | 0.75 | 0.47 | 1.00 | 0.79 | 0.75 |
| 5-symptom simple sum | 0.96 | 0.80 | 0.52 | 0.99 | 0.83 | 0.76 |
| 3-symptom predicted probabilities | 0.96 | 0.77 | 0.49 | 0.99 | 0.81 | 0.74 |
| 3-symptom simple sum | 0.96 | 0.77 | 0.49 | 0.99 | 0.81 | 0.74 |
| 30% of the sample (testing sample) | | | | | | |
| 6-symptom predicted probabilities | 0.89 | 0.79 | 0.47 | 0.97 | 0.81 | 0.68 |
| 6-symptom simple sum | 0.89 | 0.79 | 0.47 | 0.97 | 0.81 | 0.68 |
| 5-symptom predicted probabilities | 1.00 | 0.74 | 0.45 | 1.00 | 0.79 | 0.74 |
| 5-symptom simple sum | 0.89 | 0.81 | 0.50 | 0.97 | 0.83 | 0.70 |
| 3-symptom predicted probabilities | 0.89 | 0.74 | 0.42 | 0.97 | 0.77 | 0.63 |
| 3-symptom simple sum | 0.89 | 0.74 | 0.42 | 0.97 | 0.77 | 0.63 |

These results show that, on the part of the sample logistic models were fitted on, all six scores had relatively similar diagnostic abilities. Youden's J statistic values were almost the same, and all scores were set to be extremely sensitive.

However, when diagnostic analyses were conducted on the remaining part of the sample, the 30% of the sample not included in logistic analysis procedures, assessments indicated that the web-based CTS self-assessment method using 5 symptoms predicted probabilities (logistic function) and its simplified 5-symptom sum were better than assessments based on other scores, including the original CTS-6 based assessments (judging by Youden's J values).

The 3-CTS symptom score was the worst of the scores, which might be compensated by the fact that it is a much shorter method.

Of the tested scores, the web-based CTS self-assessment method using 5 symptoms, using assessment results either in the form of predicted probabilities, or simplified as a sum, seemed to best balance accuracy with length of the web-based CTS self-assessment method. The web-based CTS self-assessment method using 5 symptoms has similar J statistic values on the fitting part of the sample with the original web-based CTS-6 self-assessment method, while having better values on the testing part of the sample (30% of the sample).

### Effects of Using Weighted Sum Scores

A sum score was created that was based on assigning different weights to different symptoms instead of using simple weights. This was done separately for the 6-symptom, 5-symptom, and 3-symptom web-based CTS self-assessment methods.

The strategy for assigning weights was based on several premises:
the weights should be relatively easy to apply and calculate manually i.e., whole numbers or halves (e.g., 1.5), if necessary.
The weights should not be too different from each other.
Weights were also not completely disconnected from the results of logistic regression analyses used for predicting the CTS diagnosis in the web-based CTS self-assessment method using 6 symptoms.
Odds ratios reported in the process of binary logistic regression were used to roughly inform the assignments of weights.

The resulting distribution of score weights is provided in Table 12.

TABLE 12
Weights for the weighted sum scores, maximum scores
possible with these weights, and optimal cutoff
points based on the J statistic values

| | Weights for weighted | | |
|---|---|---|---|
| | sum scores | | |
| | 6-symptom | 5-symptom | 3-symptom |
| S1. Numbness in median | 4 | 2 | 2 |
| nerve distribution | | | |
| S2. Subjective night | 6 | 3 | 3 |
| symptoms | | | |
| S3. Thenar atrophy | 2 | 1 | N/A |
| S4. Phalen test | 5 | 3 | 4 |
| S5. Loss of 2-point | 2 | 1 | N/A |
| discrimination | | | |
| S6. Tinel sign | 1 | N/A | N/A |
| Max total | 20 | 10 | 9 |
| Optimal cut-off points | 7.5 | 3.5 | 4.5 |
| Area under the ROC curve | 0.917 | 0.916 | 0.904 |

Optimal cutoff points were calculated based on the J statistic using R v4.4.0 and the pROC package.

Weighted sum scores were created using these sums and their optimal cut-off points were calculated, followed by diagnostic characteristics using these cutoff points. Comparison of areas under the ROC curve for the three scores corresponding to the three different web-based CTS self-assessment methods evaluated indicated that this areas under the ROC curve were very similar to those obtained for predicted probability scores calculated using regression functions.

As above, accuracy levels were separately calculated on the part of the sample that was previously used for fitting the binary logistic models and on the part that was used for testing them i.e., for cross-validation. Results are presented in Table 13.

TABLE 13
Accuracy levels of the weighted sum scores using the optimal cutoff points

| | Sensitivity | Specificity | PPV | NPV | Accuracy | J |
|---|---|---|---|---|---|---|
| 70% of the sample (fitting sample) | | | | | | |
| 6-symptom weighted sum | 1.00 | 0.73 | 0.46 | 1.00 | 0.78 | 0.73 |
| 5-symptom weighted sum | 1.00 | 0.73 | 0.46 | 1.00 | 0.78 | 0.73 |
| 3-symptom weighted sum | 0.96 | 0.77 | 0.49 | 0.99 | 0.81 | 0.74 |
| 30% of the sample (testing sample) | | | | | | |
| 6-symptom weighted sum | 1.00 | 0.74 | 0.45 | 1.00 | 0.79 | 0.74 |
| 5-symptom weighted sum | 1.00 | 0.74 | 0.45 | 1.00 | 0.79 | 0.74 |
| 3-symptom weighted sum | 0.89 | 0.74 | 0.42 | 0.97 | 0.77 | 0.63 |

Results showed that:
On the fitting sample (70% of the sample), the weighted sum scores had J statistic values similar to those of simple sum scores (from Table 11).
On the testing sample (the other 30% of the full sample), the weighted sum for the web-based CTS-6 self-assessment method had a slightly higher J statistic value compared to the simple sum score and predicted probabilities (from Table 11), while J statistic values of the web-based CTS-5 and CTS-3 self-assessment methods weighted sum scores were the same (the 3-symptom score), or slightly better than the simple sum score (the 5-symptom score).

Given these results, it could likely be expected that the presented weighted sum scores, if used in web-based CTS self-assessment methods, would yield accuracy levels similar to those that could be expected from the simple sum scores or predicted probability scores based on logistic functions.

### Detailed Data

The following more detailed data was used in connection with the calculations presented in this Example 1, and provide more information regarding the relative accuracy levels and properties of the web-based CTS self-assessment methods.

Answers of patients on equivalent items of the web-based CTS self-assessment method and the in-clinic diagnosis were crosstabulated to identify true and false positives and negatives. The overall model of these crosstabulations is provided in Table 14.

TABLE 14
Model of crosstabulations used for all items

In-clinic CTS diagnosis

| | | |
|---|---|---|
| What is compared? | 0 | 1 |

| | | | |
|---|---|---|---|
| Web | 0 | True negative | False negative |
| assessment | 1 | False positive | True positive |

In Table 14, 0 denotes the zero or negative value, and 1 denotes the positive or the non-zero value, regardless of the number of points assigned to it in the scoring scheme.

Specific information for all CTS-6 indications, the Durkan pressure test and the overall CTS assessments, are presented in Tables 15-22.

TABLE 15
Crosstabulation of in-clinic diagnosis and
web-based CTS self-assessment for S1.

In-clinic
CTS diagnosis

| S1. Numbness in median nerve distribution | | 0 | 1 |
|---|---|---|---|
| Web assessment | 0 | 109 | 7 |
| | 1 | 39 | 43 |

TABLE 16
Crosstabulation of in-clinic diagnosis and
web-based CTS self-assessment for S2.

In-clinic CTS diagnosis

| | | |
|---|---|---|
| S2. Subjective night symptoms | 0 | 1 |

| | | | |
|---|---|---|---|
| Web assessment | 0 | 123 | 8 |
| | 1 | 31 | 36 |

TABLE 17
Crosstabulation of in-clinic diagnosis and
web-based CTS self-assessment for S3.

In-clinic CTS diagnosis

| | | |
|---|---|---|
| S3. Thenar atrophy | 0 | 1 |

| | | | |
|---|---|---|---|
| Web assessment | 0 | 152 | 4 |
| | 1 | 30 | 12 |

TABLE 18
Crosstabulation of in-clinic diagnosis and
web-based CTS self-assessment for S4.

In-clinic CTS diagnosis

| | | |
|---|---|---|
| S4. Phalen test | 0 | 1 |

| | | | |
|---|---|---|---|
| Web assessment | 0 | 132 | 9 |
| | 1 | 23 | 34 |

TABLE 19
Crosstabulation of in-clinic diagnosis and
web-based CTS self-assessment for S5.

In-clinic CTS diagnosis

| S5. Loss of 2-point discrimination | | 0 | 1 |
|---|---|---|---|
| Web assessment | 0 | 91 | 26 |
| | 1 | 40 | 41 |

TABLE 20
Crosstabulation of in-clinic diagnosis and
web-based CTS self-assessment for S6.

In-clinic CTS diagnosis

| | | |
|---|---|---|
| S6. Tinel sign | 0 | 1 |

| | | | |
|---|---|---|---|
| Web assessment | 0 | 157 | 6 |
| | 1 | 18 | 17 |

TABLE 21
Crosstabulation of in-clinic diagnosis and web-based
CTS self-assessment for Durkan Compression test

In-clinic CTS diagnosis

| | | |
|---|---|---|
| Durkan Compression test | 0 | 1 |

| | | | |
|---|---|---|---|
| Web assessment | 0 | 141 | 8 |
| | 1 | 22 | 27 |

TABLE 22
Crosstabulation of in-clinic diagnosis and web-
based CTS self-assessment for CTS assessments

In-clinic CTS diagnosis

| | | |
|---|---|---|
| CTS Assessment | 0 | 1 |

| | | | |
|---|---|---|---|
| Web assessment | 0 | 133 | 3 |
| | 1 | 29 | 33 |

Specific information for all six CTS indications and the Durkan pressure test, derived from in-clinic diagnoses, are presented in Tables 23-29.

TABLE 23
Crosstabulation of in-clinic diagnosis and
web-based CTS self-assessment for S1

| | | In-clinic CTS diagnosis | |
|---|---|---|---|
| S1. Numbness in median nerve distribution | | 0 | 1 |
| Web assessment | 0 | 112 | 4 |
| | 1 | 50 | 32 |

TABLE 24
Crosstabulation of in-clinic diagnosis and
web-based CTS self-assessment for S2

In-clinic CTS diagnosis

| | | |
|---|---|---|
| S2. Subjective night symptoms | 0 | 1 |

| | | | |
|---|---|---|---|
| Web assessment | 0 | 123 | 8 |
| | 1 | 31 | 36 |

TABLE 25
Crosstabulation of in-clinic diagnosis and
web-based CTS self-assessment method for S3

In-clinic CTS diagnosis

| | | |
|---|---|---|
| S3. Thenar atrophy | 0 | 1 |

| | | | |
|---|---|---|---|
| Web assessment | 0 | 152 | 4 |
| | 1 | 30 | 12 |

TABLE 26
Crosstabulation of in-clinic diagnosis and
web-based CTS self-assessment method for S4

In-clinic CTS diagnosis

| | | |
|---|---|---|
| S4. Phalen test | 0 | 1 |

| | | | |
|---|---|---|---|
| Web assessment | 0 | 132 | 9 |
| | 1 | 23 | 34 |

TABLE 27
Crosstabulation of in-clinic diagnosis and
web-based CTS self-assessment method for S5

In-clinic CTS diagnosis

| S5. Loss of 2-point discrimination | | 0 | 1 |
|---|---|---|---|
| Web assessment | 0 | 91 | 26 |
| | 1 | 40 | 41 |

TABLE 28
Crosstabulation of in-clinic diagnosis and
web-based CTS self-assessment method for S6

In-clinic CTS diagnosis

| | | |
|---|---|---|
| S6. Tinel sign | 0 | 1 |

| | | | |
|---|---|---|---|
| Web assessment | 0 | 157 | 6 |
| | 1 | 18 | 17 |

TABLE 29
Crosstabulation of in-clinic diagnosis and web-based CTS
self-assessment method for Durkan Compression Test

In-clinic CTS diagnosis

| | | |
|---|---|---|
| Durkan Compression test | 0 | 1 |

| | | | |
|---|---|---|---|
| Web assessment | 0 | 141 | 8 |
| | 1 | 22 | 27 |

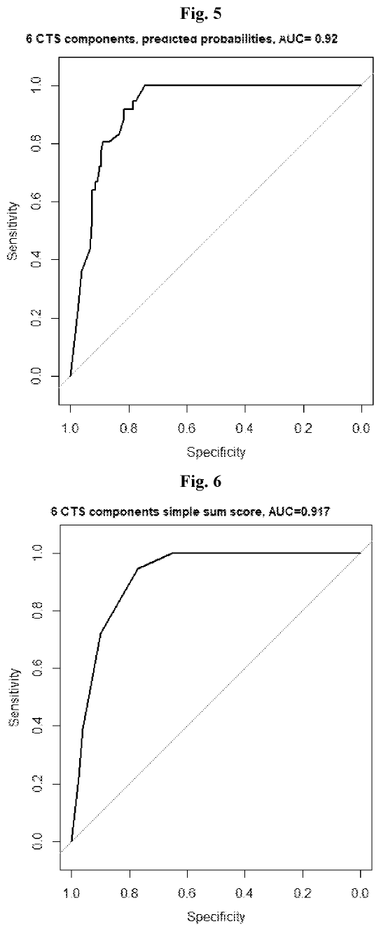

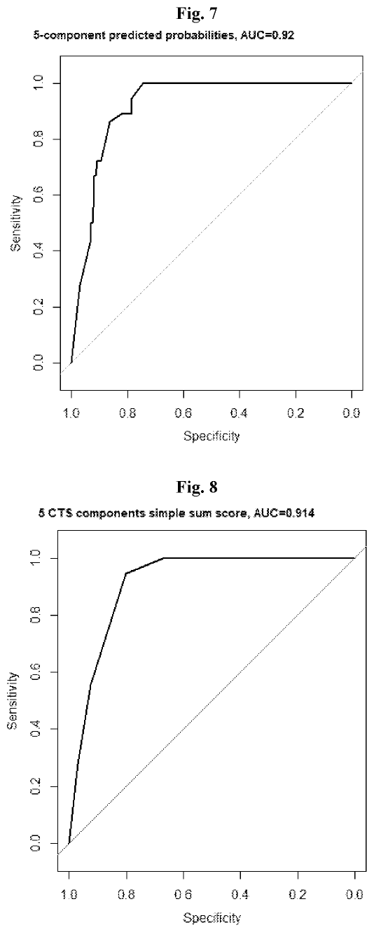

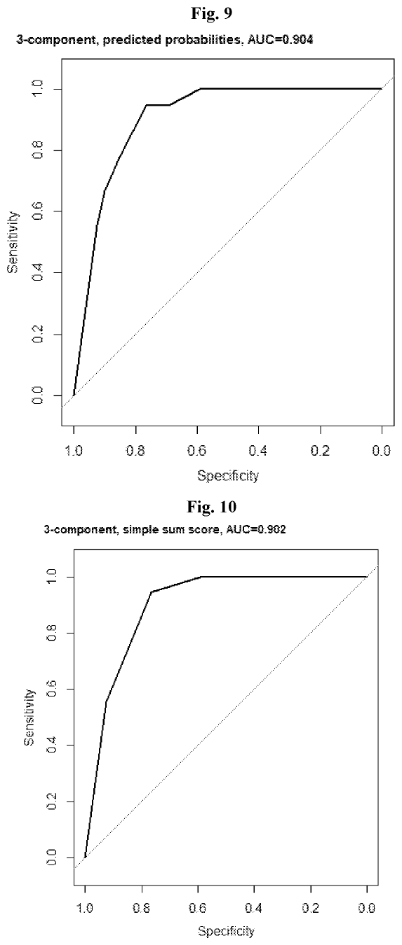

ROC curves for the all six CTS indications, the Durkan pressure test, derived from in-clinic diagnoses, are presented in FIGS. 5-10. Further information associated with these ROC curves are presented in Table 30.

TABLE 30
Crosttabulations of the scores derived from logistic
analyses procedures and binarized at their optimal
cut-off points and the in-clinic CTS diagnosis

| 70% of the sample (fitting sample) | | In-clinic CTS diagnosis | |
|---|---|---|---|
| Assessment based on: | | Negative | Positive |
| 6-symptom predicted probabilities | Negative | 92 | 1 |
| | Positive | 27 | 26 |
| 6-symptom sum | Negative | 91 | 1 |
| | Positive | 28 | 26 |
| 5-symptom predicted probabilities | Negative | 89 | 0 |
| | Positive | 30 | 27 |
| 5-symptom sum | Negative | 95 | 1 |
| | Positive | 24 | 26 |
| 3-symptom predicted probabilities | Negative | 92 | 1 |
| | Positive | 27 | 26 |
| 3-symptom sum | Negative | 92 | 1 |
| | Positive | 27 | 26 |
| 30% of the sample (testing sample) | | In-clinic CTS diagnosis | |
| Assessment based on: | | Negative | Positive |
| 6-symptom predicted probabilities | Negative | 34 | 1 |
| | Positive | 9 | 8 |
| 6-symptom sum | Negative | 34 | 1 |
| | Positive | 9 | 8 |
| 5-symptom predicted probabilities | Negative | 32 | 0 |
| | Positive | 11 | 9 |
| 5-symptom sum | Negative | 35 | 1 |
| | Positive | 8 | 8 |
| 3-symptom predicted probabilities | Negative | 32 | 1 |
| | Positive | 11 | 8 |
| 3-symptom sum | Negative | 32 | 1 |
| | Positive | 11 | 8 |
| 70% of the sample (fitting sample) | | In-clinic CTS diagnosis | |
| Assessment based on: | | Negative | Positive |
| CTS-6 total score | Negative | 95 | 1 |
| | Positive | 24 | 26 |
| 30% of the sample (testing sample) | | In-clinic CTS diagnosis | |
| Assessment based on: | | Negative | Positive |
| CTS-6 total score | Negative | 38 | 2 |
| | Positive | 5 | 7 |

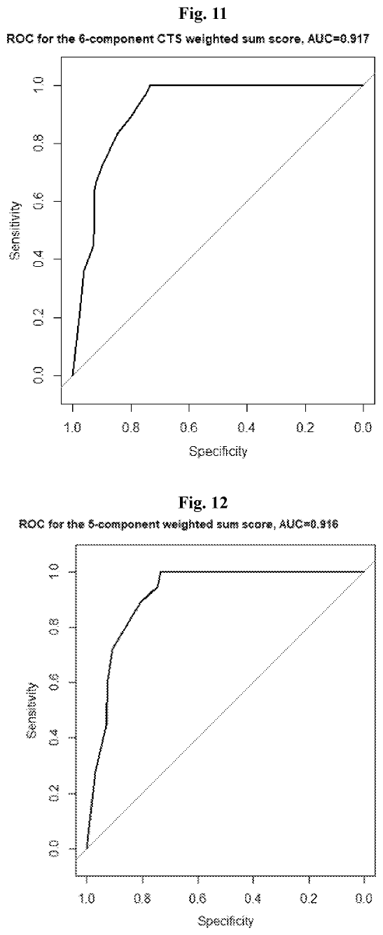

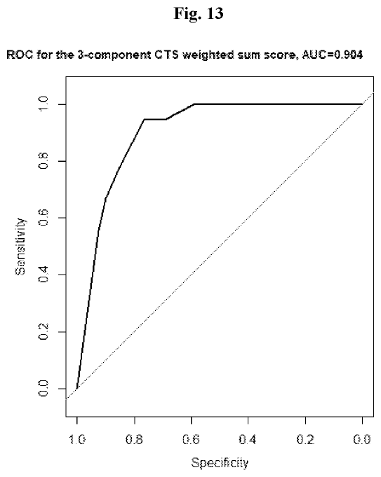

ROC curves for the weighted sum scores for web-based CTS-6, -5, and -3 self-assessment methods are presented in FIGS. 11-13. Crosstabulations associated with these ROC curves are presented in Table 31.

TABLE 31
Crosstabulations of in-clinic CTS diagnosis and assessments
produced using the weighted sum scores and the optimal cutoff
points for web-based CTS self-assessment methods

| 70% of the sample (fitting sample) | | In-clinic CTS diagnosis | |
|---|---|---|---|
| Assessment based on: | | Negative | Positive |
| 6-symptom weighted sum | Negative | 87 | 0 |
| Cutoff: 7.5 | Positive | 32 | 27 |
| 30% of the sample (fitting sample) | | In-clinic CTS diagnosis | |
| Assessment based on: | | Negative | Positive |
| 6-symptom weighted sum | Negative | 32 | 0 |
| Cutoff: 7.5 | Positive | 11 | 9 |
| 70% of the sample (fitting sample) | | In-clinic CTS diagnosis | |
| Assessment based on: | | Negative | Positive |
| 5-symptom weighted sum | Negative | 87 | 0 |
| Cutoff: 3.5 | Positive | 32 | 27 |
| 30% of the sample (fitting sample) | | In-clinic CTS diagnosis | |
| Assessment based on: | | Negative | Positive |
| 5-symptom weighted sum | Negative | 32 | 0 |
| Cutoff: 3.5 | Positive | 11 | 9 |
| 70% of the sample (fitting sample) | | In-clinic CTS diagnosis | |
| Assessment based on: | | Negative | Positive |
| 3-symptom weighted sum | Negative | 92 | 1 |
| Cutoff: 4.5 | Positive | 27 | 26 |
| 30% of the sample (fitting sample) | | In-clinic CTS diagnosis | |
| Assessment based on: | | Negative | Positive |
| 3-symptom weighted sum | Negative | 32 | 1 |
| Cutoff: 4.5 | Positive | 11 | 8 |

Overall, web-based CTS self-assessment methods using 6 CTS symptoms had a Sn/Sp of 0.92/0.90 with 84% accuracy for the assessment of CTS. The web-based CTS self-assessment method using 3 CTS indications (J=0.64) and 5 CTS indications (J=0.74) demonstrated similar accuracy levels to the 6 CTs indication version (J=0.68). Despite slight variations the performance of the individual components between studies, ≥90% Sn/Sp was demonstrated for the overall assessment, which reflects the fact that the assessment of CTS cannot be made using a single history or examination item. Accordingly, the web-based CTS self-assessment methods exhibited high levels of accuracy when using 6 CTS indications. Shorted version using 3 or 5 CTS indications demonstrated similarly high accuracy levels.

In general, more simplistic patient evaluation methods are preferable to longer or more complex methods, assuming the performance is similar. This may be of particular importance for evaluation methods that are self-administered by patients without direct physician guidance, as longer or more complex screening questions may be increasingly burdensome, especially for patients with lower health or technological literacy. In addition, these data suggest that reasonably accurate screening for CTS can also be performed by patients without direct physician guidance.

Clinical utility of the web-based CTS self-assessment methods is related to resource utilization and patient access. Pre-screening patients with referrals for suspected CTS with these web-based CTS self-assessment methods may aid in avoiding unnecessary EDS, particularly for patients who have evidence of CTS based on the web-based CTS self-assessment methods. This may be of particular value for those residing in medically underserved regions or whose insurance status makes seeking subspeciality care more difficult.

In the present description, any concentration range, percentage range, ratio range, or integer range is to be understood to include the value of any integer within the recited range, including the endpoint(s) and, when appropriate, fractions thereof (such as one tenth and one hundredth of an integer), unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include”, “have”, and “comprise” are used synonymously, which terms and variants thereof are intended to be construed as non-limiting.

The various embodiments described above can be combined to provide further embodiments. All of the non-patent publications referred to in this specification, including in the “References” below, are incorporated herein by reference, in their entirety. Aspects of the embodiments can be modified, if necessary to employ concepts of the various patents, applications and publications to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### REFERENCES

Keith M W, Masear V, Amadio P C, Andary M, Barth R W, Graham B, et al. Treatment of carpal tunnel syndrome. J Am Acad Orthop Surg. 2009; 17 (6): 397-405.
Fajardo M, Kim S H, Szabo R M. Incidence of carpal tunnel release: trends and implications within the United States ambulatory care setting. J Hand Surg Am. 2012; 37 (8): 1599e1605.
American Academy of Orthopaedic Surgeons: Management of carpal tunnel syndrome evidence-based clinical practice guideline. Available at: www.aaos.org/quality/quality-programs/upper-extremity-programs/carpal-tunnel-syndrome/. Accessed Jun. 17, 2023
Crijns T J, Mucharraz C, Paravasthuramesh A, Teunis T, Ring D, et al. Surgeons' recommendations for neurodiagnostic testing with high pretest probability of idiopathic median neuropathy at the carpal tunnel. J Hand Surg Am. 2022; 47 (8): 736-44.
Billig J I, Kotsis S V, Chung K C, Adkinson J, Fowler J, Gaston R G, et al. Variation in use of electrodiagnostic testing: analysis from the Michigan collaborative hand initiative for quality in surgery. J Hand Surg Am. 2021; 46 (3): 169-177.
Fowler J R, Cipolli W, Hanson T. A comparison of three diagnostic tests for carpal tunnel syndrome using latent class analysis. J Bone Joint Surg. 2015; 97 (23): 1958-1961.
Fowler J R, Munsch M, Tosti R, Hagberg W C, Imbriglia J E. Comparison of ultrasound and electrodiagnostic testing for assessment of carpal tunnel syndrome: study using a validated clinical tool as the reference standard. J Bone Joint Surg. 2014; 96 (17): e148.
Graham B, Regehr G, Naglie G, Wright J G. Development and validation of diagnostic criteria for carpal tunnel syndrome. J Hand Surg Am. 2006; 31:919-924.
Graham B. The value added by electrodiagnostic testing in the assessment of carpal tunnel syndrome. J Bone Joint Surg. 2008; 90 (12): 2587-2593.
Grandizio L C, Boualam B, Shea P, Hoehn M, Cove C, Udoeyo I F, et al. The reliability of the CTS-6 for examiners with varying levels of clinical experience. J Hand Surg Am. 2022; 47 (6): 501-506.
Grandizio L C, Ozday Y, Mettler A W, Garcia C V, Manzar S, Akoon A, et al. Sensitivity, specificity and reliability of the CTS-6 for carpal tunnel syndrome administered by medical assistants. J Hand Surg Am. Accepted for Publication April 2024.
Grandizio L C, Barreto-Rocha D, Foster B K, Udoeyo I F. Evaluation of a comprehensive telemedicine pathway for carpal tunnel syndrome: a comparison of virtual and in-person assessments. J Hand Surg Am. 2022; 47 (2): 111-119.
Billig J I, Sears E D. Utilization of diagnostic testing for carpal tunnel syndrome: a survey of the American Society for Surgery of the Hand. J Hand Surg Am. 2022; 47 (1): 11-18.
Greenfield P T, Spencer C C, Dawes A, Wagner E R, Gottschalk M B, Daly C A. The preoperative cost of carpal tunnel syndrome. The Journal of Hand Surgery. 2022 Aug. 1; 47 (8): 752-61.
Sears E D, Swiatek P R, Hou H, Chung K C. Utilization of preoperative electrodiagnostic studies for carpal tunnel syndrome: an analysis of national practice patterns. J Hand Surg Am. 2016; 41 (6): 665-672.
Smith M M, Tremont G, Ott B R. A review of telephone-administered screening tests for dementia assessment. Am J Alzheimers Dis Other Demen. 2009; 24 (1): 58-69.
Sears E D, Lu Y T, Wood S M, Nasser J S, Hayward R A, Chung K C, Kerr E A. Diagnostic testing requested before surgical evaluation for carpal tunnel syndrome. The Journal of hand surgery. 2017 Aug. 1; 42 (8): 623-9.
Abrahamyan L, Sahakyan Y, Chung S, Pechlivanoglou P, Bielecki J, Carcone S M, Rac V E, Fitzpatrick M, Krahn M. Diagnostic accuracy of level I V portable sleep monitors versus polysomnography for obstructive sleep apnea: a systematic review and meta-analysis. Sleep and Breathing. 2018 September; 22:593-611.
Baker W F, Kwan S A, Radack T, Rivlin M. Use of Telemedicine Among Hand Surgeons and Their Patients. The Journal of Hand Surgery. 2024 Jan. 1; 49 (1): 23-7.
Orman O, Uzun B A, Aydoğdu A, Baydar M, Pehlivanoğlu G. Evaluation of Injured Structures and Circulation of Fingers From Photos Taken in the Emergency Department After Hand Injury. The Journal of Hand Surgery. 2024 Aug. 22.
Rizzi A M, Polachek W S, Dulas M, Strelzow J A, Hynes K K. The new ‘normal’: Rapid adoption of telemedicine in orthopaedics during the COVID-19 pandemic. Injury. 2020 Dec. 1; 51 (12): 2816-21.
Tadley M, Henry T W, Horan D P, Beredjiklian P K. The financial implications of telehealth visits within a hand and wrist surgery clinical practice during the COVID-19 pandemic. The Journal of Hand Surgery. 2021 Aug. 1; 46 (8): 660-5.
Scott K L, Skotak C M, Renfree K J. Remote assessment of wrist range of motion: inter- and intra-observer agreement of provider estimation and direct measurement with photographs and tracings. The Journal of Hand Surgery. 2019 Nov. 1; 44 (11): 954-65.
Tinwala H, Brinkman N, Ramtin S, Ring D, Crijns T, Reichel L. Factors Associated With Comfort Using Telemedicine for Upper Limb Specialty Care. The Journal of Hand Surgery. 2023 Jul. 1; 48 (7): 647-54.
Axell-House D B, Lavingia R, Rafferty M, Clark E, Amirian E S, Chiao E Y. The estimation of diagnostic accuracy of tests for COVID-19: A scoping review. Journal of Infection. 2020 Nov. 1; 81 (5): 681-97.
Wang G, Fiedler A K, Warth R J, Bailey L, Shupe P G, Gregory J M. Reliability and accuracy of telemedicine-based shoulder examinations. Journal of Shoulder and Elbow Surgery. 2022 Aug. 1; 31 (8): e369-75.
Kennedy C E, Yeh P T, Gholbzouri K, Narasimhan M. Self-testing for pregnancy: a systematic review and meta-analysis. BMJ open. 2022 Feb. 1; 12 (2): e054120.
Lu A D, Veet C A, Aljundi O, Whitaker E, Smith W B, Smith J E. A systematic review of physical examination components adapted for telemedicine. Telemedicine and e-Health. 2022 Dec. 1; 28 (12): 1764-85.
Bradley K E, Cook C, Reinke E K, Vinson E N, Mather III R C, Riboh J, Lassiter T, Wittstein J R. Comparison of the accuracy of telehealth examination versus clinical examination in the detection of shoulder pathology. Journal of Shoulder and Elbow Surgery. 2021 May 1; 30 (5): 1042-52.
Davis G M, Spanakis E K, Migdal A L, Singh L G, Albury B, Urrutia M A, Zamudio-Coronado K W, Scott W H,
Doerfler R, Lizama S, Satyarengga M. Accuracy of Dexcom G6 continuous glucose monitoring in non-critically ill hospitalized patients with diabetes. Diabetes Care. 2021 Jul. 1; 44 (7): 1641-6.
Tiron R, Lyon G, Kilroy H, Osman A, Kelly N, O'Mahony N, Lopes C, Coffey S, McMahon S, Wren M, Conway K. Screening for obstructive sleep apnea with novel hybrid acoustic smartphone app technology. Journal of Thoracic Disease. 2020 August; 12 (8): 4476.
Shaukat A, Levin T R. Current and future colorectal cancer screening strategies. Nature reviews Gastroenterology & hepatology. 2022 August; 19 (8): 521-31.
Geyer R E, Kotnik J H, Lyon V, Brandstetter E, Suchsland M Z, Han P D, Graham C, Ilcisin M, Kim A E, Chu H Y, Nickerson D A. Diagnostic accuracy of an at-home, rapid self-test for influenza: prospective comparative accuracy study. JMIR Public Health and Surveillance. 2022 Feb. 22; 8 (2): e28268.
Freeman K, Dinnes J, Chuchu N, Takwoingi Y, Bayliss S E, Matin R N, Jain A, Walter F M, Williams H C, Deeks J J. Algorithm based smartphone apps to assess risk of skin cancer in adults: systematic review of diagnostic accuracy studies. bmj. 2020 Feb. 10; 368.
Van Nest D S, Ilyas A M, Rivlin M. Telemedicine evaluation and techniques in hand surgery. Journal of hand surgery global online. 2020 Jul. 1; 2 (4): 240-5.
Johnson C, Taff K, Lee B R, Montalbano A. The rapid increase in telemedicine visits during COVID-19. Patient Experience Journal. 2020; 7 (2): 72-9.
Kim C Y, Wiznia D H, Wang Y, Save A V, Anandasivam N S, Swigart C R, Pelker R R. The effect of insurance type on patient access to carpal tunnel release under the Affordable Care Act. The Journal of Hand Surgery. 2016 Apr. 1; 41 (4): 503-9.
Kuroiwa T, Sarcon A, Ibara T, Yamada E, Yamamoto A, Tsukamoto K, Fujita K. The potential of ChatGPT as a self-diagnostic tool in common orthopedic diseases: exploratory study. Journal of medical Internet research. 2023 September 15; 25: e47621.
Ardakani A A, Afshar A, Bhatt S, Bureau N J, Tahmasebi A, Acharya U R, Mohammadi A. Assessment of carpal tunnel syndrome: A comparative study of shear wave elastography, morphometry and artificial intelligence techniques. Pattern Recognition Letters. 2020 May 1; 133:77-85.
Yener Y, Darol E S, Uzun S, Alemdar M, çolak T. Application of Machine Learning Algorithms for Carpal Tunnel Syndrome. Journal of the Anatomical Society of India. 2024 Apr. 1; 73 (2): 178-83
Hageman M G, Anderson J, Blok R, Bossen J K, Ring D. Internet self-assessment in hand surgery. Hand. 2015 September; 10 (3): 565-9.
Baer L, Jacobs D G, Cukor P, O'Laughlen J, Coyle J T, Magruder K M. Automated telephone screening survey for depression. J Am Med Assoc. 1995; 273 (24): 1943-1944.
Rocca W A, Maraganore D M, McDonnell S K, Schaid D J. Validation of a telephone questionnaire for Parkinson's disease. J Clin Epidem. 1998; 51 (6): 517-23.
Landis J R, Koch G G. An application of hierarchical kappa-type statistics in the assessment of majority agreement among multiple observers. Biometrics 1977; 33:363-374.
Lindenhovius A, Karanicolas P J, Bhandari M, Ring D, Collaborative C O. Radiographic arthrosis after elbow trauma: interobserver reliability. J Hand Surg Am. 2012; 37 (4): 755-9.
Bujang M A, Adnan T H. Requirements for minimum sample size for sensitivity and specificity analysis. J Clin Diagn Res. 2016; 10 (10): e1-6.
Fujita K, Watanabe T, Kuroiwa T, Sasaki T, Nimura A, Sugiura Y. A tablet-based app for carpal tunnel syndrome screening: diagnostic case-control study. JMIR mHealth and uHealth. 2019; 7 (9): e14172.
Koyama T, Sato S, Toriumi M, Watanabe T, Nimura A, Okawa A, Sugiura Y, Fujita K. A screening method using anomaly detection on a smartphone for patients with carpal tunnel syndrome: Diagnostic case-control study. JMIR mHealth and uHealth. 2021; 9 (3): e26320.
Riley C O, Mckinstry B, Fairhurst K. Accuracy of telephone screening tools to identify dementia patients remotely: systematic review. JRSM Open. 2022; 13 (9): 20542704221115956.
Muskens E M, Lucassen P, Groenleer W, Van Weel C, Oude Voshaar R, Speckens A. Psychiatric assessment by telephone: is it an opportunity?. Soc Psychiatry Psychiatr Epidemiol 2014; 49:1677-1689.
de Figueiredo F P, Parada A P, Cardoso V C, Batista R F, da Silva A A, Barbieri M A, et al. Postpartum depression screening by telephone: a good alternative for public health and research. Arch Womens Ment Health. 2015; 18:547-553.
Sears E D, Lu Y T, Wood S M, Nasser J S, Hayward R A, Chung K C, Kerr E A. Diagnostic testing requested before surgical evaluation for carpal tunnel syndrome. The Journal of hand surgery. 2017 Aug. 1; 42 (8): 623-9.
Burrus M T, Werner B C, Starman J S, Kurkis G M, Pierre J M, Diduch D R, et al. Patient perceptions and current trends in internet use by orthopedic outpatients. HSS Journal. 2017; 13 (3): 271-275.
Rozental T D, George T M, Chacko A T. Social networking among upper extremity patients. J Hand Surg. 2010; 35 (5): 819-823.
United States Department of Health and Human Services. The Health Insurance Portability and Accountability Act of 1996 (HIPPA) Privacy, Security and Breach Notification Rules. Available at: http://www.hhs.gov/ocr/privacy/. Accessed Mar. 10, 2019
United States Department of Health and Human Services. Available at: http://www.hhs.gov/ocr/privacy/hipaa/administrative/omnibus/. Accessed Mar. 10, 2019
Naam N H, Sanbar S. Advanced technology and confidentiality in hand surgery. J Hand Surg. 2015; 40 (1): 182-187.
Friebe I, Isaacs J, Mallu S, Kurdin A, Mounasamy V, Dhindsa H. Evaluation of appropriateness of patient transfers for hand and microsurgery to a level I trauma center. HAND. 2013; 8 (4): 417-421.
Petkovic D, Wongworawat M D, Anderson S R. Factors affecting appropriateness of interfacility transfer for hand injuries. HAND. 2018; 13 (1): 108-113.
Tripod M, Tait M, Bracey J, Sexton K, Beck W, Wyrick T O. The use of telemedicine decreases unnecessary hand trauma transfers. HAND. Published Online: Nov. 12, 2018 (doi: 10.1177/1558944718810877).
Buvik A, Bugge E, Knutsen G, Smábrekke A, Wilsgaard T. Quality of care for remote orthopaedic consultations using telemedicine: a randomized controlled trial. BMC Health Serv Res. 2016; 16 (1): 483.
Wagner E R, Conti Mica M, Shin A Y. Smartphone photography utilized to measure wrist range of motion. J Hand Surg Eur. 2018; 43 (2): 187-192.
Zhao J Z, Blazar P E, Mora A N, Earp B E. Range of motion measurements of the fingers via smartphone photography. HAND. Published Online: Jan. 28, 2019 (doi: 10.1177/1558944718820955 Meislin M A, Wagner E R, Shin A Y. A comparison of elbow range of motion measurements: smartphone-based digital photography versus goniometric measurements. J Hand Surg. 2016; 41 (4): 510-55.
Tofte J N, Anthony C A, Polgreen P M, Buckwalter J A, Caldwell L S, Fowler T P, et al. Postoperative care via smartphone following carpal tunnel release. J Telemed Telecare. 2018; November 14:1357633X18807606.
Abel K C, Baldwin K, Chuo J, Ganley T, Kim A, Wells L, Giordano T. Can telemedicine be used for adolescent postoperative knee arthroscopy follow-up? JBJS J Orthop Phys Assist. 2017; 5 (4): e26.
Harno K, Arajärvi E, Paavola T, Carlson C, Viikinkoski P. Clinical effectiveness and cost analysis of patient referral by videoconferencing in orthopaedics. J Telemed Telecare. 2001; 7 (4): 219-225.
Ohinmaa A, Vuolio S, Haukipuro K, Winblad I. A cost-minimization analysis of orthopaedic consultations using videoconferencing in comparison with conventional consulting. J Telemed Telecare. 2002; 8 (5): 283-289.
Buvik A, Bergmo T S, Bugge E, Smaabrekke A, Wilsgaard T, Olsen J A. Cost-effectiveness of telemedicine in remote orthopedic consultations: randomized controlled trial. J Med Internet Res. 2019; 21 (2): e11330.
Sathiyakumar V, Apfeld J C, Obremskey W T, Thakore R V, Sethi M K. Prospective randomized controlled trial using telemedicine for follow-ups in an orthopedic trauma population: a pilot study. J Orthop Trauma. 2015; 29 (3): e139-145.
Buvik A, Bugge E, Knutsen G, Småbrekke A, Wilsgaard T. Patient satisfaction with remote orthopaedic consultation by using telemedicine: A randomized controlled trial. J Telemed Telecare. Published Online: Jul. 4, 2018 (doi: 10.1177/1357633X18783921).
De La Torre-Díez I, López-Coronado M, Vaca C, Aguado J S, de Castro C. Cost-utility and cost-effectiveness studies of telemedicine, electronic, and mobile health systems in the literature: a systematic review. Telemed e-Health. 2015 Feb. 1; 21 (2): 81-85.

1. A computer-implemented method of guideline-directed self-assessment of carpal tunnel syndrome (CTS) using a set of CTS symptoms, the method comprising:
for each CTS symptom in the set of CTS symptoms:
displaying a CTS symptom self-assessment guideline and prompting a patient to perform the guideline and observe results;
displaying information regarding how the results correlate with whether the patient does or does not exhibit the CTS symptom and prompting input of a yes/no indicator correlating with whether the patient exhibits the CTS symptom;
receiving and storing an input indicator for the CTS symptom in a set of CTS symptom indicators, wherein the input indicator has a value of 1 for a yes indicator and a value of 0 for a no indicator;
determining a CTS assessment for the patient, based on a comparison of a sum of the values of the CTS indicators to a cutoff point; and
displaying the CTS assessment to the patient or transmitting the CTS value to a physician associated with the patient.

2. The method of claim 1, wherein the set of CTS symptoms comprises between 3-6 symptoms.

3. The method of claim 2, wherein the CTS symptoms comprise or consist of: numbness in median nerve distribution; subjective night symptoms; thenar atrophy; phalen test; loss of 2-point discrimination; or tinel sign, and any combinations thereof.

4. The method of claim 2, wherein the CTS symptoms comprise or consist of: numbness in median nerve distribution; subjective night symptoms; thenar atrophy; phalen test; or loss of 2-point discrimination, and any combinations thereof.

5. The method of claim 2, wherein the CTS symptoms comprise or consist of: numbness in median nerve distribution; subjective night symptoms; or phalen test, and any combinations thereof.

6. The method of claim 1, wherein the guideline comprises instructions for performing a physical evaluation; answering a question regarding current or recent physical state without the need for a physical evaluation; or observing results, and any combinations thereof.

7. The method of claim 1, wherein the CTS assessment is yes/no assessment correlating with whether the patient does or does not have CTS.

8. The method of claim 1, wherein the CTS assessment is a graduated assessment correlating with likelihoods that the patient has CTS.

9. The method of claim 1, wherein the sum of the values of the CTS indicators is a simple sum of the values of the set of CTS symptom indicators.

10. The method of claim 1, wherein the sum of the values of the CTS indicators is a weighted sum of the values of the set of CTS symptom indicators.

11. The method of claim 1, wherein the CTS assessment is determined using a method that has an accuracy based on ROC of at least 75% when compared to in-clinic CTS diagnoses by physicians with CTS expertise.

12. The method of claim 1, wherein the CTS assessment is determined using a method that has a sensitivity/specificity of at least 0.85 when compared to in-clinic CTS diagnoses by physicians with CTS expertise.

13. The method of claim 1, wherein the CTS assessment is determined using a method that has a J value of at least 0.6 when compared to in-clinic CTS diagnoses by physicians with CTS expertise.

14. The method of claim 1, wherein the method further comprises, based on the CTS assessment correlating with the patient not having CTS, displaying a further medical care recommendation comprising a recommendation that further medical care for CTS is not needed.

15. The method of claim 1, wherein the method further comprises, based on the CTS assessment correlating with the patient having CTS or having a likelihood of having CTS above a selected level, displaying a further medical care recommendation comprising a recommendation that the patient seek professional medical care for CTS.

16. The method of claim 1, further comprising displaying a referral recommendation comprising information regarding a medical professional with expertise in CTS;
requesting permission to transmit the CTS assessment and patient-identifying information to at least one medical professional from the referral recommendation;
receiving an input granting or denying such permission; and
if such permission is granted, transmitting the CTS assessment and patient-identifying information to the medical professional(s).

17. A method of diagnosis of CTS in a patient by a physician, the method comprising:
obtaining a CTS assessment of the patient, wherein the CTS assessment is generated by the method of claim 1; and
based on the CTS assessment, undertaking a diagnostic or therapeutic action.

18. The method of claim 17, wherein the diagnostic action comprises a physical examination of the patient by a medical professional; assessment of the CTS symptoms using the CTS assessment by or under direction of the medical professional; or diagnostic testing, and any combinations thereof, or wherein the therapeutic action comprises surgery to alleviate CTS; referral to another physician with expertise in CTS, or both.

19. A method of seeking treatment or diagnosis of CTS by a patient, the method comprising:
obtaining a CTS assessment of the patient, wherein the CTS assessment is generated by the method of claim 1; and
based on the CTS assessment, determining whether to seek further medical care.

20. A system for guideline-directed self-assessment of carpal tunnel syndrome (CTS) using a set of CTS symptoms, the system comprising:
an electronic device comprising a processor, memory, display, input, and I/O interface capable of storing instructions within the memory that, when executed by the processor, cause the electronic device to perform or communicate via the I/O interface over a network with a patient data processing center that includes a processor, a memory and an I/O interface, and cause the patient data processing center to perform a self-administered CTS assessment method according to claim 1; or
a patient data processing center comprising a processor, a memory, and an I/O interface capable of storing instructions within the memory that, when executed by the processor cause the patient data processing center to perform or communicate via the I/O interface over a network with an electronic device containing a processor, memory, display, and input and cause the electronic device to perform a self-administered CTS assessment method according to claim 1.
